(19) United States
(12) Patent Application Publication
(10) Pub. No.: US 2024/0082533 A1
(43) Pub. Date: Mar. 14, 2024

(54) SYSTEM FOR FUNCTIONAL REHABILITATION AND/OR PAIN REHABILITATION DUE TO SENSORIMOTOR IMPAIRMENT

(71) Applicant: SENSEFUL TECHNOLOGIES AB, GÖTEBORG (SE)

(72) Inventors: Max Jair ORTIZ CATALAN, Göteborg (SE); Enzo MASTINU, Mölndal (SE); Mirka BUIST, Göteborg (SE)

(73) Assignee: SENSEFUL TECHNOLOGIES AB, GÖTEBORG (SE)

(21) Appl. No.: 18/275,440

(22) PCT Filed: Feb. 11, 2022
(86) PCT No.: PCT/SE2022/050148

§ 371 Date: Aug. 2, 2023

(30) Foreign Application Priority Data
Feb. 12, 2021 (SE) 2150154-9

(51) Int. Cl. A61M 21/00; A61M 19/00; G06F 3/01; G16H 40/63

(52) U.S. Cl. CPC A61M 21/00; A61M 19/00; G06F 3/015; G06F 3/016; G16H 40/63; A61M 2021/0022; A61M 2021/0066; A61M 2205/04; A61M 2205/3317; A61M 2230/60

(57) ABSTRACT

The present invention generally relates to a system for functional rehabilitation and/or pain rehabilitation due to sensorimotor impairment, comprising: an input system for providing control to a user to perform a task in a training environment; and at least one actuator or electrode placeable in contact with a user's body part for providing somatosensory stimulation to the user based on an output from the training environment related to the performed task.

### FIELD OF THE INVENTION

The present invention generally relates to a system for functional rehabilitation and/or pain rehabilitation due to sensorimotor impairment, to a control unit configured to control a training environment for functional rehabilitation and/or pain rehabilitation due to sensorimotor impairment, to a corresponding computer program product and to a method for functional rehabilitation and/or pain rehabilitation due to sensorimotor impairment.

### BACKGROUND

The inability to move or perceive sensations (sensorimotor impairment) can result in the experience of pain in the impaired part of the body. Such impairment can be caused by amputation, nerve injuries, or stroke. The resulting pain is in nature neuropathic and is also known as phantom limb pain (PLP) or stroke pain, depending on the etiology.

Various treatments have been proposed to cure PLP. One example is pharmacological treatments that are meant to manage rather than cure the condition and often carry considerable side effects.

Another example is surgical intervention techniques, such as Targeted Muscle Reinnervation (TMR). However, patients treated with surgical techniques, such as TMR, still report PLP (T. Kuiken et al., Targeted Muscle Reinnervation, vol. 20131732. Taylor &amp; Francis, 2013). Further, surgical interventions may cause complications and inconvenience for the patient.

The use of powered prosthesis has been found correlated with lower incidences of PLP (M. Lotze et al. Nat. Neurosci., vol. 2, no. 6, pp. 501-2, June 1999), yet most patients still report PLP, and some cannot even wear a prosthesis because of PLP.

Mirror Therapy is a common treatment for PLP and similar neuropathic pains, such as complex regional pain syndrome (CPRS). In this treatment, a patient who has lost one limb places the contralateral, still able limb in front of a mirror. This creates the visual illusion that the missing limb is still there. The patient is then asked to perform movements that reflect as movements by the missing limb. The success rate for mirror therapy varies, and further, mirror therapy is not possible for contralateral amputees.

Accordingly, there is room for improvements with regards to systems and methods for treating conditions caused by sensorimotor impairment.

### SUMMARY

In view of the above-mentioned and other drawbacks of the prior art, it is an object of the present invention to provide an improved system for functional rehabilitation and/or pain rehabilitation due to sensorimotor impairment that at least partly alleviates the drawbacks of prior art.

According to a first aspect of the invention, there is provided a system for functional rehabilitation and/or pain rehabilitation due to sensorimotor impairment.

The system comprises an input system for providing control to a user to perform a task in a training environment; and at least one actuator or electrode placeable in contact with a user's body part for providing somatosensory stimulation to the user based on an output from the training environment related to the performed task.

The user's body part may be adjacent to an affected body part, or be adjacent to the location of a missing body part in the case of amputees. Further, the body part may be an affected body part itself, thus the somatosensory stimulation may be provided to an affected body part, for example in the case of nerve injures or stroke. Thus, the somatosensory stimulation is intended to be provided to an affected body part or to a body part directly linked with the affected body part or linked with a previous location of a now missing body part.

Generally, a body part is herein meant to be any body part excluding the brain. In other words, embodiments disclosed herein does not relate to brain-computer interfaces.

An affected body part should be broadly interpreted to include a body part that is in some way affected by sensorimotor impairment. For example, the affected body part may have been affected by e.g. nerve injuries, or stroke and may be e.g. paralyzed.

The tasks performed by the user may be one or more of motor, sensory, and/or sensorimotor tasks. For example, the training tasks may be a combination of motor tasks, sensory tasks, and sensorimotor tasks. The training tasks may also be one of motor, sensory, and sensorimotor tasks.

The present invention is at least partly based on the realization that providing somatosensory stimulation to a body part related to an affected body part, or directly to the affected body part of the user in response to the outcome of a task performed by the user provides an effective way to treat conditions caused by sensorimotor impairment. The inventors realized that by purposely engaging the affected sensorimotor neural circuitry, this becomes disengaged from pain processing and thus resulting in pain relief.

The present invention is further based on the realization that a more user-friendly and even portable device is obtained by the present system that relies on somatosensory stimulation provided by actuators and/or electrodes placeable on the skin of the user's body part. Such a portable device can for example be wearable by attachment to the body of the user with e.g., straps, a sleeve or a belt, or adhesives such as a double-sided adhesive strip.

Further this system is more accessible to users compared to e.g. transcranial magnetic stimulation (TMS), surgical interventions, and pharmacological treatments that also often has disadvantageous side effects.

The training environment is the “space” or “infrastructure” or “framework” or “ecosystem” where the task is performed by the user using the input system. Thus, using the input system, the user affects one or more elements of the training environment, whereby an output is provided from the training environment. This output is translated to somatosensory stimulation provided to the user's body part by the at least one actuator or electrode. The training environment may be a virtual environment including video games, but it may also be a tangible environment such as including mechanical elements or robotic elements controlled by the user using the input system.

The output from the training environment may be directly or indirectly related to input provided by the user to the training environment for performing the task. As an example, the output may be directly related to the input in the case of using e.g. biofeedback. As a further example, the output may be indirectly related to the input in the case of output to reflect consequences on the training environment produced by the user's input. In both cases, the output from the training environment is determined based on the input provided by the user when performing the task. A tactile output provided by the at least one actuator may form a tactile representation of the training environment similar to that of pixels of a display.

In other embodiments the output may be related to sounds of the training environment, e.g., music or special effects. As an example, the output may be related to sounds from a video of the training environment where the output is related to the intensity of the sound, or the output may be related to the beat of music. In this embodiment the system may comprise a microphone to record the sound and process it into an adequate output. In possible implementations of embodiments of the present invention in the form of a portable device, the user may wear the at least one actuator or electrode while watching television and receive somatosensory feedback related to the sound of the television.

The input system may comprise of one or more of e.g. a keyboard, or a mouse, or other devices that the user can use to control elements of the training environment. Generally, the input system is adapted to give the user full control of at least one element of the training environment such that the user can perform a task in the training environment. In other words, the input system provides motor control in the training environment to the user.

A user may herein be considered a patient using the system or methods. The patient may be in need of sensorimotor impairment rehabilitation.

In a preferred embodiment, the input system may comprise a set of sensors for collecting signals from a user's body part indicative of intended motions of an affected body part or a missing body part for performing the task in the training environment; and a processing circuitry configured to operate a motor volition decoder for decoding the signals to infer the intended motions, and to control at least one element of the training environment based on the decoded signals. As discussed above, the body part is not intended to be the brain. Thus, the sensors are not intended to collect signals from the surface of a user's skull to thereby collect signals directly from the brain indicative or neural activity. The sensors are preferably configured to collect signals from a body part adjacent to an affected body part, or be adjacent to the location of a missing body part in the case of amputees, or from the affected body part itself.

Thus, the set of sensors may collect signals from a user's body part being adjacent to an affected body part or being the affected body part itself or adjacent a missing body part, indicative of intended motions of the affected body part or the missing body part for performing the task in the training environment.

Motor neural circuitry may be maximally engaged by allowing the user to execute complex movements i.e. simultaneous motions in more than one joint of the affected body part, for example a limb, decoded via neuromuscular models or machine learning algorithms fed with the user's bioelectric signals. Such bioelectric signals may be e.g. myo- or neuro-electric signals from the spinal cord or peripheral nerves, not from the brain directly via detectors such as electrodes arranged on or near the user's skull.

The motor control may be achieved by means of motor volition decoders, independently or in conjunction, with conventional human-machine interfaces such as one or more of a keyboard, a mouse, a touchscreen, and a joystick.

Thus, in some embodiments, the motor volition decoder may be configured to decode the signals and infer the intended motions by the user according to a model such as a machine learning model. The model may be trained using prior learning data.

The machine learning models can be but not exclusively Artificial Neural Networks, Support Vector Machines, or k-Nearest Neighbors, as well as other probabilistic algorithms such as Gaussian Mixture Model or Linear Discriminant Analysis. Both supervised and unsupervised machine learning algorithms are conceivable for embodiments of the present invention.

The sensors may be electrodes arrangeable on a user's skin for recording electric signals.

In other embodiments, the sensors may be electrodes that are implantable into the user's body for recording electric signals. In this way, user control of the training element may be provided via a brain computer interface.

In some embodiments, the system may advantageously comprise an array of actuators and/or electrodes placeable on the user's body part for providing the somatosensory stimulation. Sensory neural circuitry is maximally engaged by providing rich somatosensory stimulation such as e.g. vibration, pressure, stroke, strain, and touch. This is advantageously accomplished using an array of actuators and/or electrodes to provide a high-definition tactile matrix.

Further types of actuators include pressure and/or sliding bands, and tendon vibrators. With this, the motor and sensory tasks may be coordinated in exercises designed for mindful motor and sensory engagement which provides improved pain relief, and due to the mindful sensorimotor engagement, further functional gains such as improved motor control and sensory perception.

In some embodiments the array of actuators and/or electrodes is comprised in a haptic display.

The array of actuators may advantageously provide a tactile representation of the training environment to the user.

The at least one actuator or electrode placeable in contact with a user's body part for providing somatosensory stimulation may be placeable on the user's body, such as on the surface of the skin of the body part.

In embodiments, the at least one electrode for providing the somatosensory stimulation may comprise electrodes implantable into the user's body part.

In some embodiments, the system may comprise a kinesthetic feedback device configured to provide kinesthetic feedback to the user's body based on the outcome of the decoded signals. There are different options to create kinaesthetic feedback. One option is to vibrate tendons to create the illusion that the connected muscle moves. A further kinesthetic feedback device includes electrodes for electrical stimulation to activate proprioceptive fibres. Another option is to apply strain to the skin to provide kinaesthetic information. Generally, this allows for providing an even richer somatosensory stimulation with a resulting improved pain relief and improved sensory perception.

There are various ways to provide further feedback from the training environment as an output to further improve the rehabilitation performance provided by the system.

For example, in one embodiment the system may comprise a display for providing visual feedback from the training environment. The visual feedback may be a visual representation of the training environment. Thus, in the visual feedback, the user can see the elements and objects and their movements or other consequences caused by the input provided by the user.

In one embodiment, the system may comprise an audio device for providing auditory feedback to the user based on a further output from the training environment. The audio feedback may be for enhancing some actions in the training environment. For example, user input may cause a consequence in the training environment which is fed back as for example an audio signal to the user.

In one embodiment, the system may comprise a thermal device for providing thermal feedback to the user based on a further output from the training environment. Thus, in order to further enhance some consequences or actions in the training environment, thermal feedback may be provided.

In one embodiment, the system may comprise an exoskeletal stabilization unit arrangeable to stabilize the motion of the user's body part.

The exoskeletal stabilization unit may be powered to assist the user in moving the body part when performing the task. In this way, further kinesthetic feedback may be provided by actively moved joints, instead of a movement illusion provided by some other kinesthetic feedback devices. It further provides for assisting when muscles of a user do not receive enough neural drive to actuate a joint as in cases of e.g. stroke and spinal cord injury.

In one embodiment, the system may comprise a brain modulation and/or brain stimulation device configured to excite, inhibit, and/or stimulate the user's brain. The brain modulation and/or stimulation device may be configured to provide modulation or stimulation actions while the user is performing a task in the training environment, and/or while the system is providing somatosensory stimulation to the user. The brain modulation and/or stimulation device may be configured to provide modulation or stimulation actions once the user has finished a task in the training environment, and/or while the system has completed providing the associated somatosensory stimulation to the user. Example devices that may provide brain modulation and/or stimulation, include devices that can provide transcranial electrical stimulation (TES) such as transcranial Direct Current Stimulation (tDCS), transcranial Alternating Current Stimulation (tACS), transcranial Random Noise Stimulation (tRNS), transcranial Pulsed Current Stimulation (tPCS). Further example devices that may provide brain modulation and/or stimulation is Transcranial Magnetic Stimulation (TMS) devices.

According to a second aspect of the invention, there is provided a control unit configured to control a training environment for functional rehabilitation and/or pain rehabilitation due to sensorimotor impairment, the control unit being configured to: receive user input related to the user performing a task in a training environment, the user input being receivable from an input system configured to provide control to a user for performing the task; control at least one element for affecting the task in the training environment based on the user input; process variables of the training environment effected by the performed task into an output to at least one actuator or electrode placeable on/in the user's body for providing somatosensory stimulation.

Examples of process variables are the location or movement, e.g. direction and velocity, of an object in the training environment, the density or area of an object, the pattern or shape of an object. The variables described the present state of the training environment.

Such process variables may be calculated by the control unit configured to track elements of the training environment in a coordinate system.

In embodiments, the control unit may be configured to: receive signals collected from a user's body part by a set of sensors, the signals being indicative of intended motions of an affected body part or a missing body part for performing the task in the training environment; decode the signals to thereby infer the intended motions, and control the at least one element for affecting the task in the training environment based on the decoded signals.

Further effects and features of the second aspect of the invention are largely analogous to those described above in connection with the first aspect of the invention.

According to a third aspect of the invention, there is provided a computer program product comprising a computer readable medium having stored thereon computer program means for controlling a training environment for functional and pain rehabilitation due to sensorimotor impairment, wherein the computer program product comprises: code for processing user input related to performing a task in a training environment, the user input being receivable from an input system configured to provide control to a user for performing the task; code for controlling at least one element for affecting the task in of the training environment based on the user input; and code for processing variables of the training environment effected by the performed task into an output to at least one actuator or electrode placeable on/in the user's body for providing somatosensory stimulation.

In embodiments, the computer program product may comprise code for decoding signals collected from a user's body part by a set of sensors, the signals being indicative of intended motions of an affected body part or a missing body part for performing the task in the training environment, code for controlling at least one element of the training environment based on the decoded signals; and code for processing variables of the training environment affected by the performed task into an output to at least one actuator or electrode placeable on/in the user's body part for providing somatosensory stimulation.

Further effects and features of the third aspect of the invention are largely analogous to those described above in connection with the first aspect and the second aspect of the invention.

According to a fourth aspect of the invention, there is provided a method for functional rehabilitation and/or pain rehabilitation due to sensorimotor impairment comprising: receiving user input related to performing a task in a training environment from an input system configured to provide control to a user for performing the task in the training environment; transforming at least one element of the training environment based on the user input; and providing somatosensory stimulation to a user's body part using at least one actuator or electrode, the somatosensory stimulation being based on an output from the training environment related to the performed task.

In embodiments, the method may comprise: collecting signals from the user's body part indicative of intended motions of an affected body part or a missing body part for performing the task in the training environment; decoding the signals to infer the intended motions, transforming at least one element of the training environment based on the decoded signals; and providing somatosensory stimulation to the user's body part using at least one actuator or electrode, the somatosensory stimulation being based on an output from the training environment related to the performed task.

In embodiments, the method may comprise providing progressively more difficult task to the user depending on the outcome of the prior performed task.

In embodiments, the method may comprise providing brain modulation or brain stimulation to the user while the user is performing a task in the training environment, and/or while the system is providing somatosensory stimulation to the user. Advantageously, brain stimulation provides a means of providing sensory feedback that improves the rehabilitation performance provided by the system. Further, brain modulation advantageously provides a way to modulation, e.g., reduce activation thresholds for targeted neural circuits to enhance learning from the training tasks, facilitate performing the tasks, and generally improve the efficiency of the rehabilitation performance provided by the system.

The brain modulation or brain stimulation may be triggered by an event in the training environment similarly to other feedback types, such as for example the user completing or initiating a task, the user interacting with the task environment, e.g., touching an object, or performing movements in the training environment, or a more peripheral event part of the training environment that is affecting the task performed or to be performed, e.g., background items or sounds in the training environment. In some implementations, the location of brain modulation/stimulation can be changed according to particular task type, for example depending on whether the task is motor, sensory and/or sensorimotor. Moreover, the brain modulation/stimulation can be turned on/off accordingly to promote and/or inhibit the activation of neural circuits.

In some implementations the brain modulation and/or brain stimulation may be provided in parallel to performing the task in the training environment. In this case, the brain modulation and/or brain stimulation is provided independently of events or actions in the training environment and is thus not triggered by such events.

In embodiments, the method may comprise providing brain modulation or brain stimulation to the user once the user is completed in performing a task in the training environment. and/or in response to that the somatosensory stimulation has been provided to the user.

In embodiments, the method may comprise providing brain modulation or brain stimulation prior to the user performing a task in the training environment, and/or in response to that the somatosensory stimulation has been provided to the user.

In embodiments, the brain modulation or brain stimulation may be provided to the user independent of the task performed by the user in the training environment. Thus, brain modulation or stimulation may be used independent of a specific task. For example, brain modulation or stimulation may be turned on before starting and stay active during and in between a variety of training tasks, e.g., sensory, motor and/or sensorimotor, and even stay on after the whole training session is finished, without being triggered by a specific task. Purely as an example, a training session may include: start brain modulation/stimulation such as tDCS as brain stimulation, perform motor training/tasks for a time duration e.g., 20 minutes, subsequently, perform sensory training/tasks for a time duration e.g., 20 minutes, subsequently perform sensory motor training/tasks for a time duration e.g., 30 minutes, subsequently, stop brain modulation/stimulation e.g., tDCS.

Example devices that may provide brain modulation and/or stimulation, include tDCS, tACS, tRNS, tPCS, TMS, TES, or similar.

Further effects and features of the fourth aspect of the invention are largely analogous to those described above in connection with the first aspect, the second aspect, and the third aspect of the invention.

According to a fifth aspect of the invention, there is provided a method for controlling a sensorimotor impairment rehabilitation system, comprising: receiving user input related to performing a task in a training environment from an input system configured to provide control to a user for performing the task in the training environment; transforming at least one element of the training environment based on the user input; and providing somatosensory stimulation to a user's body part using at least one actuator or electrode, the somatosensory stimulation being based on an output from the training environment related to the performed task.

Further effects and features of the fifth aspect of the invention are largely analogous to those described above in connection with the first aspect, the second aspect, and the third aspect, and the fourth aspect of the invention.

Further features of, and advantages with, the present invention will become apparent when studying the appended claims and the following description. The skilled person realize that different features of the present invention may be combined to create embodiments other than those described in the following, without departing from the scope of the present invention.

### BRIEF DESCRIPTION OF THE DRAWINGS

These and other aspects of the present invention will now be described in more detail, with reference to the appended drawings showing an example embodiment of the invention, wherein:

FIG. 1 is a box-diagram of a system for functional rehabilitation and/or pain rehabilitation due to sensorimotor impairment according to embodiments of the present invention;

FIG. 2 conceptually illustrates a system according to embodiments of the present invention;

FIG. 3 is a functional box-diagram of creating motion volition input in a system according to embodiments of the present invention;

FIG. 4 is a box-diagram of various types of sensory feedback that may be provided to the user of the system according to embodiments of the present invention;

FIG. 5 is a box diagram showing a system overview according to embodiments of the present invention;

FIG. 6 conceptually illustrates a system according to embodiments of the present invention;

FIG. 7 illustrates an example of a training environment and corresponding sensory feedback according to embodiments of the present invention;

FIG. 8 illustrates an example of a training environment and corresponding sensory feedback according to embodiments of the present invention;

FIG. 9 illustrates an example of a training environment and corresponding sensory feedback according to embodiments of the present invention;

FIG. 10A conceptually illustrates a system according to embodiments of the present invention;

FIG. 10B conceptually illustrates a system according to embodiments of the present invention;

FIG. 10C conceptually illustrates a system according to embodiments of the present invention;

FIG. 10D conceptually illustrates a system according to embodiments of the present invention;

FIG. 11A conceptually illustrates a user using a system according to embodiments of the present invention;

FIG. 11B conceptually illustrates an exoskeleton unit according to embodiments of the present invention;

FIG. 11C conceptually illustrates an exoskeleton unit according to embodiments of the present invention;

FIG. 12 conceptually illustrates a user using a system according to embodiments of the present invention;

FIG. 13 conceptually illustrates a user using a system according to embodiments of the present invention;

FIG. 14 is a flow-chart of method steps according to embodiments of the present invention;

FIG. 15 is a flow-chart of method steps according to embodiments of the present invention;

FIG. 16 is a table of different difficulty levels of a task with corresponding types of user controls and feedback;

FIG. 17 schematically illustrates an example of a game with progressively increasing difficulty levels;

FIG. 18A is a box-diagram schematically illustrating how a difficulty level is related to the various types of outputs provided as feedback to the user;

FIG. 18B is a box-diagram schematically illustrating how a difficulty level is related to the various types of outputs provided as feedback to the user;

FIG. 18C is a box-diagram schematically illustrating how a difficulty level is related to the various types of outputs provided as feedback to the user;

FIG. 18D is a box-diagram schematically illustrating how a difficulty level is related to the various types of outputs provided as feedback to the user;

FIG. 18E is a box-diagram schematically illustrating how a difficulty level is related to the various types of outputs provided as feedback to the user;

FIG. 19A conceptually illustrates a haptic feedback arrangement according to embodiments of the present invention; and

FIG. 19B conceptually illustrates a haptic feedback arrangement attached to a user's body part.

### DETAILED DESCRIPTION OF EXAMPLE EMBODIMENTS

In the present detailed description, various embodiments of the present invention are herein described with reference to specific implementations. In describing embodiments, specific terminology is employed for the sake of clarity. However, the invention is not intended to be limited to the specific terminology so selected. While specific exemplary embodiments are discussed, it should be understood that this is done for illustration purposes only. A person skilled in the relevant art will recognize that other components and configurations can be used without parting from the scope of the invention.

FIG. 1 is a box-diagram of a system 100 for functional rehabilitation and/or pain rehabilitation due to sensorimotor impairment according to embodiments of the present invention. The system 100 comprises an input system 102 for providing control to a user to perform a task in a training environment 104.

The system 100 further comprises at least one actuator 106 or electrode 106 placeable in contact with a user's body part for providing somatosensory stimulation to the user based on an output from the training environment 104 related to the performed task.

The input system 102 enables for the user to provide input signals to the training environment to control elements of the training environment to solve a task. The input signals are processed by processing circuitry such as a control unit 108 in accordance with the performed task. Depending on the outcome of the processing, i.e. the outcome of the performed task, is corresponding output signals provided to the at least one actuator 106 or electrode 106. The output is translated to somatosensory stimulation by the at least one actuator 106 or electrode 106.

The input system 102 provides motor control to the user in the training environment 104. There are several conceivable input systems that are applicable to embodiments of the present invention, example input systems comprise motion volition encoders, and human-machine interfaces such as keyboards, mouses, touchscreens, and joysticks.

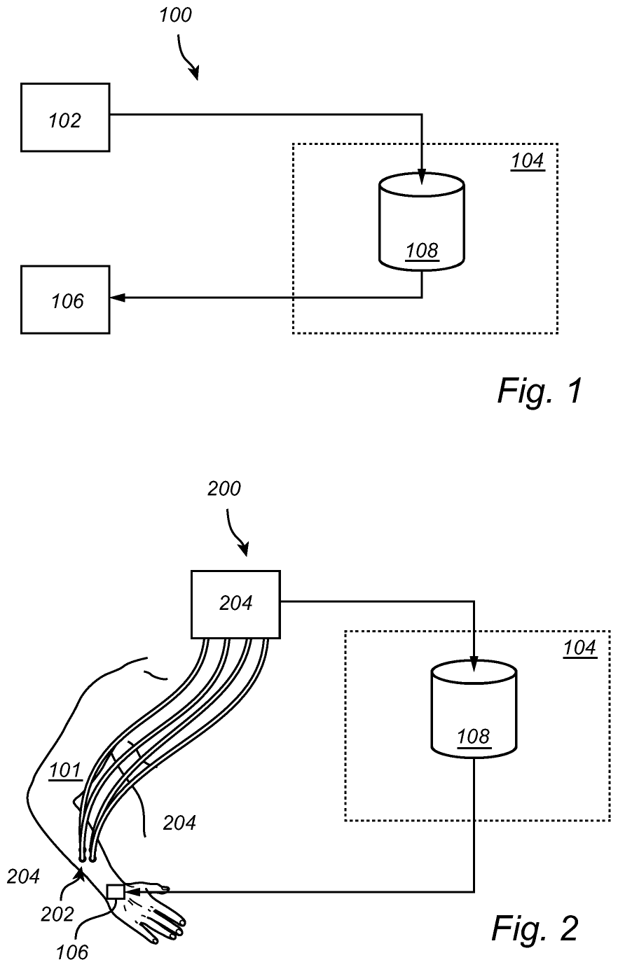

FIG. 2 conceptually illustrates a system 200 according to embodiments of the present invention. The system 200 comprises an input system comprises a set of sensors 202 for collecting signals from the user's body part 101. The signals are indicative of intended motions of an affected body part or a missing body part, where the intended motions are intended for performing the task in the training environment 104, as discussed in conjunction with FIG. 1. Further, processing circuitry, e.g. a control unit 204 is configured to operate a motor volition decoder for decoding the signals to infer the intended motions, and to control at least one element of the training environment 104 based on the decoded signals. In other words, the processing circuitry is wirelessly, or via wires, able to receive the signals recorded by the sensors 202, decode the signals to infer the motions that the user intents to perform with an affected or missing body part.

Advantageously, the motor volition decoders allow the user to be in control of a particular task while sensory feedback relevant to that task is provided to the user, predominantly by haptic feedback via e.g. the at least one actuator 106 or electrode 106. In other words, the motor volition decoders provide input to the training environment while e.g. haptic actuators constitute its output. For this the system comprises an electronic interface represented by 204 and 108 in FIG. 2, including the motion decoder configured to translate the input provided by the user to the training environment to an output as effected by the task currently performed. In this way, the user's motor volition is translated into inputs that effect the training environment on which the task is being performed, and the consequence of the input is feedback to the user. In other words, feedback is produced by translating the state of the variables in the training environment into outputs to the feedback actuators or electrodes.

The sensors are preferably electrodes arrangeable on a user's skin for recording electric signals, so-called electromyographic signals. However, the sensors may in some embodiments be electrodes that are implantable into the user's body for recording electric signals.

Other sensors that are conceivable include pressure sensors forming so-called pressure maps, or inertial measurement signals.

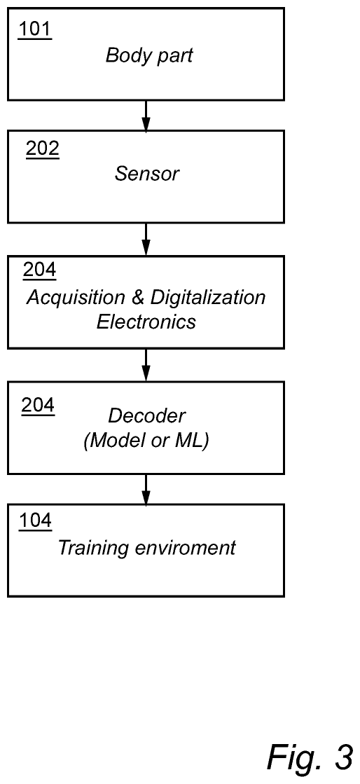

The motor volition decoder operated by the processing circuitry is advantageously configured to decode the signals and infer the intended motions by the user according to prior training of a model. This will be discussed in more detail with reference to FIG. 3.

FIG. 3 is a functional box-diagram of creating motion volition input in a system 200 according to embodiments of the present invention. The sensors 202 collect e.g., bioelectrical signals such as EMG, or pressure maps or inertial measurements from the body part 101. Acquisition and digitalization electronics 204 receives and samples the collected signals and provides filtered and digitalized signals to a motion decoder, here included in 204, that may operate to decode the signals and infer the intended motions by the user according to prior training of a model. The outcome of the decoding is processed such that at least one element of the training environment 104 is controlled based on the decoded signals.

With regards to the sensors, in the case of electromyographic (EMG) signals, conventional passive and/or active surface electrodes can be applied to the surface of the user's skin so to capture the electric activity generated by muscular contractions. Multiple electrodes can be used together as to create an array for simultaneous recording as used in machine learning approaches.

In addition, electromyographic and electroneurographic signals from invasive sources, i.e., implanted electrodes, can additionally be acquired, provided basic and safe compatibility between connectors and acquisition electronics.

In the case of pressure signals, a matrix of Force Sensitive Resistor (FSR) sensors can be applied to the surface of the user's skin so to capture the pressure distribution patterns generated by muscular contractions.

In the case of inertial signals, Inertial Measuring Units (IMUs) including accelerometers, gyroscopes and magnetometers can be applied to the targeted body part of the user so to capture motion activity generated by translation and rotation. The IMUs can be applied directly on the user's skin and/or within elastic bands wore on the targeted body part. Multiple electrodes can be used together as to create a joint-map for simultaneous recording as conventionally used in neuromuscular modelling and machine learning approaches.

The acquisition and digitalization electronics may include suitable analog filtering depending on the nature of the signals to be acquired. The filters may include conventional high-pass for DC and artifacts rejection, notch for power line noise rejection and low-pass for smoothing and anti-aliasing purposes. In addition, adaptive filtering solutions may be applied. Depending on the implementation at hand, the filters can include basic RC components as well as low-noise operational amplifiers. The analog filtering part may also incorporate components that can provide basic electrical safety to the user and the electronics such as diodes for blocking Electrostatic Discharge (ESD) events to/from the user, capacitors for blocking direct current flowing to the user, and resistors and capacitors to limit any leakage current flowing to the user.

Moreover, signal amplifiers may be included to enable suitable interfacing with the signal to be acquired. The design and configuration of the amplifier depends on the nature of the target signal. The amplifiers may for example be used independently or in combination, as to adapt to the electrodes and sensors in use. For example, monopolar and/or bipolar amplifiers can be used. If hardware-differential acquisition (i.e., bipolar amplifier) is not available, a software-differential solution can be implemented if deemed sufficient for the present implementation.

When multi-channel acquisition is required, this can for example be achieved via dedicated amplifiers per channel or via time-multiplexing a single amplifier. Integrated analog-front-end solutions are preferred; however, any other custom solution is also possible whenever a higher customization of parameters is required. Parameters such as gain, dynamic range and bandwidth are adapted to the signal of interest and to the included modules of sampling and digitalization electronics.

In the case of surface EMG signals, a dedicated reference electrode is usually applied on an electrically neutral part of the body (e.g., boney points like wrist or elbow joint). For invasive EMG signals, the reference can be either on an implanted electrode or on a superficial one.

In the case of pressure signals, the FSR matrix of sensors is usually referenced to the ground of the electronics and no extra applied electrodes are required. The same often applies to inertial signals too.

Analog-to-digital converters (ADC) and a central processing unit (CPU), i.e. processing circuitry, are preferably used to finalize the digitalization of the signals of interest. This step allows the following software module to process the data and ultimately infer the motor volition of the user. The ADC may or may not be included in the CPU. In the latter case, the ADC is interfaced by the CPU for signal readout via conventional e.g., SPI, USART and I2C, or custom serial protocols. In the ADC, parameters such as resolution and sampling frequency are adapted to the signal of interest for the implementation at hand. In the case of EMG signals, a resolution above or equal to 16 bits is preferred. The sampling frequency is matched, via Nyquist theorem, to a typical bandwidth of interest of [0+1000] Hz. Oversampling, noise shaping, and decimation techniques can be used to increase the accuracy of the measurements.

In the case of pressure and inertial signals, there is a lower demand in matter of resolution and sampling frequency, where resolutions above or equal to 12 bit and frequencies above or equal to 150 Hz are usually appropriate.

Similarly, to the amplifier's module, integrated analog-front-end solutions are preferred. In such scenario the amplification and digitalization stages can both be handled by a single integrated chip capable of communication with external devices. This chip is then interfaced by the CPU for parameters calibration and signal readout.

A decoder receives the sampled and processed signals from the acquisition and digitalization electronics. In the decoder, machine learning or probabilistic approaches, particularly pattern recognition and regression algorithms, are applied for decoding the motor intent from EMG, pressure, and inertial signals. The main assumption of such approaches is that each movement, whether it is from an intact or a “phantom” limb, is characterized by reproducible muscular contractions that can be described by a set of features. The features represent each movement as a distinct cluster in the features space, facilitating the decoding task of the algorithm.

It is understood that, an electrophysical meaningful relation between these features and the actual signals recorded from the user is not necessary or relevant, as long as it proves to be robust enough for a consistent decoding. The features can be defined in the time, in the frequency and in the time-frequency domains. Some examples of the features include: mean absolute value, zero crossing, waveform length, slope changes, median, variance, standard deviation, correlation, co-variance, autoregressive coefficients, wavelet coefficients, root mean square, power, max fractal length, fractal dimension, entropy, Wilson amplitude, mean of amplitude. These features can all be used independently or in combination as to adapt to the processed signals and/or to the user.

The features are extracted from time fragments (i.e., time windows) of the recorded signals whose length can usually vary from 50 to 250 milliseconds. In addition, an overlap between neighboring time windows may be used.

The decoding output can be discrete (e.g., classification, pattern recognition algorithms) or continuous (e.g., regression algorithms). The decoding algorithms can be classified as supervised and unsupervised depending if it needs pre-labelled data as training samples, and both can be inspired by statistics (e.g., naïve Bayes, linear discriminant analysis), logic (e.g., decision tree), biology (e.g., artificial neural networks) and geometry (e.g., support vector machine). For classification tasks, single and/or multiple decoding algorithms are configured for a single and/or multiple classes, constituting different algorithm topologies such as single, all-movements-as-individual, ago-antagonist-mixed, one-vs-all, one-vs-one, and all-and-one. Depending on the topology in use, the decoding output allows for individual and/or simultaneous motions. In the case of simultaneous motions, multiple degrees of freedom can be operated at the same time coherently with multiple intentions of the user (e.g., flex elbow and close the hand).

Neuromusculoskeletal models can also be used to estimate human body kinematics from the recorded muscular contractions, optionally also in conjunction with inertial and other sensorial measurements. For these approaches, the prediction target is set on the intended joint angle to be reached rather than which particular movement to be performed. Neuromusculoskeletal models can operate directly from the EMG signal or on the motor unit discharge rates, which sometimes is estimated from the EMG. In both cases, an accurate kinematic model for the body part of interest is required.

The stream of decoding output can be further processed to filter out potential erroneous predictions. For this scope, post-prediction algorithms are utilized such as majority voting buffer, velocity-based decision ramp, filtering and smoothing.

After the robustness and correctness of the decoding output, the next priority is set on the proportionality of these output to the actual muscular contraction level deployed by the user. Being able of generating proportional motor control output can facilitate the embodiment of such human-machine interface. The proportionality can come inherently from regressive approaches or it can be calculated by comparison to known reference value for minimum and maximum strength (or speed), or it can be predicted by another machine learning algorithm dedicated to this task.

The motor control achieved by means of motor volition decoders can also be used, independently or in conjunction, with conventional human-machine interfaces such as keyboard, mouse, touchscreen, and joystick.

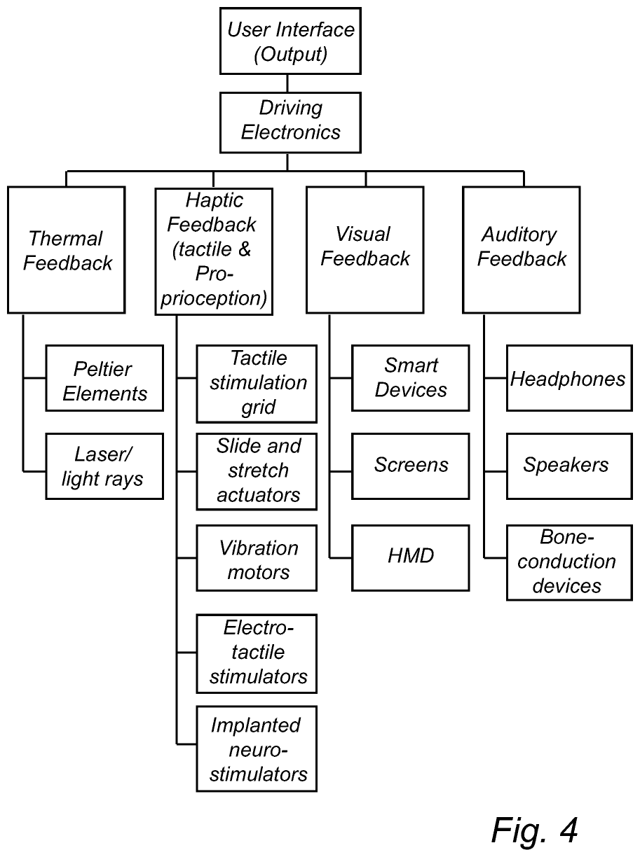

FIG. 4 is an overview of various types of sensory feedback that may be provided to the user of the system according to embodiments of the present invention. Output signals are provided from the training environment, the output signals being directly or indirectly related to input provided by the user to the training environment for performing the task. The output signals cause a driving electronics to provide feedback to the user via one or more feedback devices.

Thus, sensory feedback may be provided to the user using more than one feedback device, where the sensory feedback or somatosensory feedback provided by the more than one feedback device is based on the output from the training environment. Accordingly, consequences in the training environment caused directly or indirectly by user input, may be translated by a control unit to somatosensory and/or sensory feedback to the user via more than one feedback device, as conceptually illustrated in FIG. 4 where several types of feedback devices are exemplified, including thermal feedback devices, haptic feedback devices, visual feedback devices, and auditory feedback devices.

For example, the system may comprise an array of actuators and/or electrodes placeable on the user's body part for providing the somatosensory stimulation. Such actuators and electrodes correspond to devices for providing haptic feedback. For example, the array of actuators and/or electrodes may be a haptic display.

Haptic feedback may be provided as tactile and electro-tactile stimuli combination. An actuator stimulates mechanoreceptors of the body part, while an electrode stimulates the nerve producing distally referred sensations in the body part.

Further, the at least one electrode for providing the somatosensory stimulation may comprise electrodes implantable into the user's body part, e.g. implantable neurostimulators.

According to embodiments of the present invention, tactile feedback may advantageously be provided in more than one way, because different mechanisms activate different tactile sensations. These are activated by activating different mechanoreceptors.

One such mechanoreceptors is Meissner's corpuscles that are activated by e.g.: ultrasonic waves, tactile points created by e.g. magnetic actuators in an array, acoustic/elastomeric/ferro fluidic displays, dielectric elastomer actuators, pressure valve actuators, electro tactile displays, or any other mechanism where a point is applied to the skin, in accordance with embodiments of the present invention. For example, actuators placed in an array to form an array of tactile points create a tactile/haptic display. This display is placed against the skin of the body part (that is the body part that is trained). The display can be used to depict pixels or objects of a game. The vibration can be used to alert the player of an in-game event.

Such actuators may be implemented in various way where one conceivable implementation is by means of an array of magnetic actuators, where each tactile point, e.g. each actuator, comprises a coil with a magnetic core. By actuating the coil, the magnetic core moves in and out of the coil, creating a tactile point when it touches the skin of the user's body part.

One such mechanoreceptors is Merkel's disks that are activated by e.g.: vibration actuators on low frequencies in accordance with embodiments of the present invention.

One such mechanoreceptors is Pacinian corpuscles that are activated by e.g.: vibration actuators on high frequencies and pressure force generated by e.g. motors, bands, pulleys, pneumatics, or hydraulics in accordance with embodiments of the present invention.

One such mechanoreceptors is Hair follicle plexus that are activated by e.g.: sliding actuators e.g. a slider, or an airflow, in accordance with embodiments of the present invention. For example, a rope or string, wrapped around the arm, attached to a continuous servomotor on each of the ends of the string. By actuating both servo motors in the same direction, a sliding sensation (hair follicle plexus) is achieved. Adding e.g. a knot or other fixed element, e.g. a ball or pearl, or similar, in the rope, contributes to the possibility of identifying a single position on the axis of the string. By actuating both servo motors in opposite directions a pressure sensation (Pacinian corpuscle) is achieved. An in-game application of a training environment may be to match the position of one of the sliders with the other, or to indicate the position of two in-game elements compared to each other.

One such mechanoreceptors is Ruffini's endings that are activated by e.g.: mechanisms that stretch or strain the skin in accordance with embodiments of the present invention. For example, a rope or string laid on top of the skin, in the direction of the muscle fibers, connected to a servo motor on one side, and to a fixed point on the other side. By actuating the servo motor, the rope is tightened creating a sense of skin stretch (Ruffini's endings), creating the illusion of proprioception i.e., alternating joint configurations. An in-game application of a training environment may be to mimic e.g., g-forces on the body in a racing game, or to indicate the direction of an object.

Somatosensation can also be provided via electrical stimulation. The use of electrical stimulation can be achieved via both non-invasive (e.g., skin electrodes) and invasive (e.g., implanted electrodes) solutions. For both types, it is possible to elicit distally referred sensations via neural stimulation (e.g. stimulation in a proximal location resulting in a sensation perceived in a more distally located part of the body).

In some possible implementations, the system comprises a display for providing visual feedback from the training environment. Such displays may be desktop displays, smart devices such as mobile phones or tablets or smartwatches, laptops, computer displays, head mounted displays, etc. Visual feedback advantageously provides for displaying the training/rehabilitation tasks (e.g., games or exercises), as well as the results of the user's motion volition (e.g., motor commands in real-time or otherwise). It can also be used to monitor the progress of the training.

Further, the system may comprise an audio device for providing auditory feedback to the user based on a further output from the training environment. Such audio devices may be e.g., speakers, headphones, earbuds, bone conduction devices, etc. Auditory feedback may be used for principal or supplementary guidance through the training.

Auditory feedback may be modulated based on the current interactions in the training environment, or be special effects on games to stimulate engagement. It could also take over the functionality of the visual or somatosensory feedback by verbally explaining task to be performed by the user. The audio feedback may be used to provide a combination between giving guidance to the training and stimulating the user.

Further, the system may comprise a thermal device for providing thermal feedback to the user based on a further output from the training environment. Thermal feedback entails two types of sensations, warm and cold. For the sensation of heat, an option is to apply lasers or light sources. Peltier Elements are an alternative option for both warm and cold sensations. Thermal fluids are another option to apply heat or cold sensations to a surface.

The system may comprise kinesthetic feedback device configured to provide kinesthetic feedback, so called tactile proprioception, to the user's body based on the outcome of the decoded signals. Kinaesthetic feedback may be used to train the proprioceptive sense. Proprioceptive feedback may be given according to input EMG signals. This can be advantageously useful for users who for example have difficulties with muscle activation, are unable to bend joints, have too weak muscles, or have had an amputation. When motor signals are sent, but the user is unable to bend the joint, the kinaesthetic feedback can be used as an alternative confirmation that the user activated the right signals. It is not necessary to connect the kinaesthetic to input EMG signals, it can be an individual feedback element independent of the input EMG signals. Creating the sense of muscle movement can be used for some games or training purposes.

Conceivable devices for providing kinesthetic feedback include tendon vibrators and to create the illusion that the connected muscle moves. Electrical stimulation to activate proprioceptive fibres is also an option. Another further possible implementation is to apply strain to the skin to provide kinaesthetic information. Besides creating movement illusions, an alternative option is to actively move joints with for example active exoskeletons.

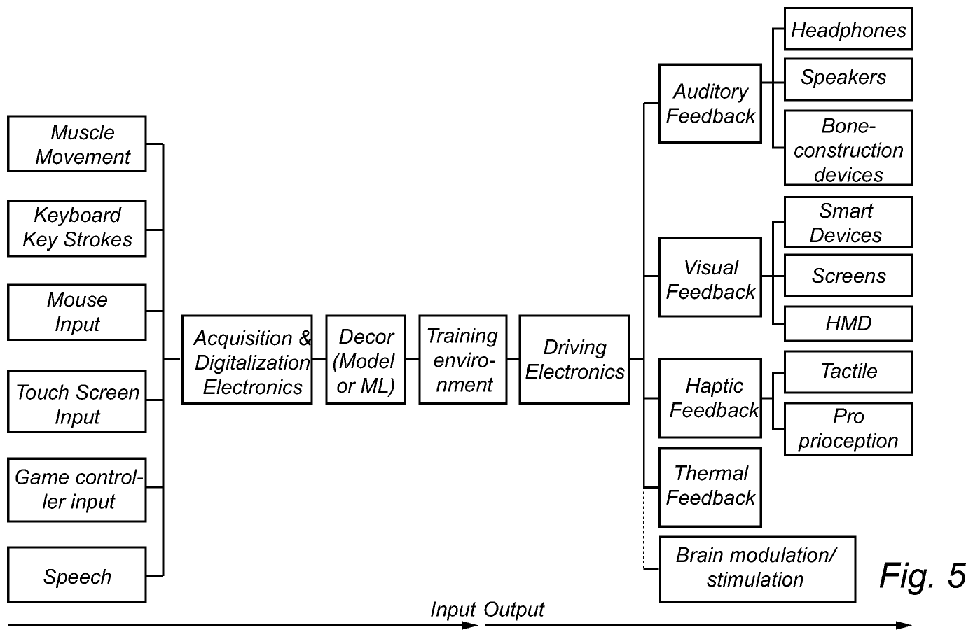

FIG. 5 is a box diagram showing a system overview according to embodiments of the present invention. On the input side, various input systems are schematically represented such as systems recording muscle movement, keyboards, mouse, touch screen, game controllers, and speech. One or more of these input systems may be used in embodiments of the present invention.

The input system(s) is/are connected to the electronic interface described with reference to FIG. 3, including the acquisition and digitalization electronics, and a decoder. On the output side of the training environment, driving electronics control one or more of a set of feedback systems as discussed in relation with FIG. 4, including at least one of auditory feedback, visual feedback, haptic feedback, and thermal feedback.

In some embodiments, the system is also configured to provide brain modulation signals and/or brain stimulation signals to a user's brain as indicated on the output side of the system in FIG. 5. This allows for to excite, inhibit, and/or stimulate the user's brain while performing a task in the training environment, before performing a task or after a task is completed. The brain modulation and/or stimulation may be triggered by events in the training environment via the drive electronics. However, the modulation and/or stimulation may in other implementations be provided independently of events in the training environment and in such case may be operated by its own drive electronics, independent of the driving electronics that controls the one or more of the other feedback systems.

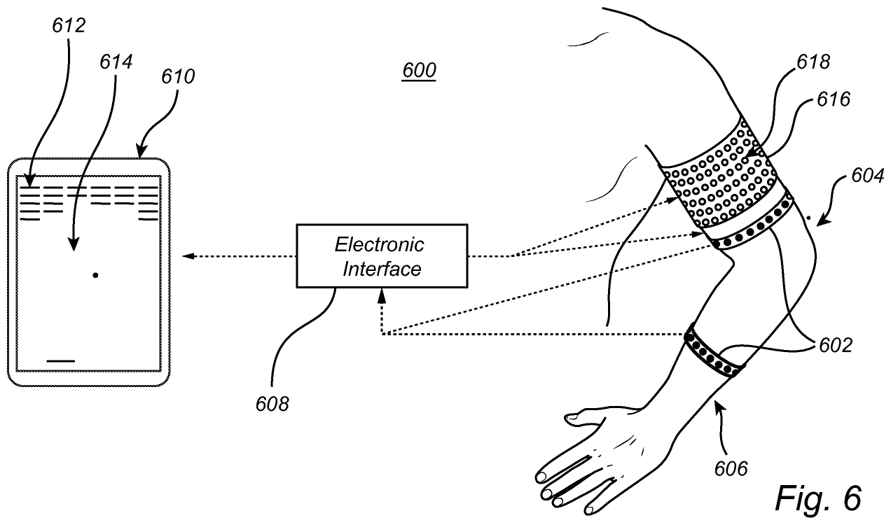

FIG. 6 conceptually illustrates a system 600 according to embodiments of the present invention. The system 600 comprises a set of sensors preferably in the form of electrodes 602 arrangeable on the surface of the user's skin on here on the limb 604. The electrodes 602 collect signals indicative of intended motions of an affected limb 606 adjacent to the body part 604 where the electrodes 602 are placed. The affected limb 606 is affected due to sensorimotor impairment. For example, the affected limb 606 may be a paralyzed limb.

The collected signals are processed by processing circuitry 608 to decode the collected signals using a motion volition decoder operative to infer the intended motions of the affected limb 606. The processing circuitry 608 translates the signals collected by the electrodes 618 to movements of an element in a training environment 610, here illustrated in the form of a game played on an electronic device 612 comprising a display 614 to visually represent the game.

Further, the system 600 comprises an array 616 of actuators and/or electrodes placeable on the user's body part for providing the somatosensory stimulation. For example, the array 616 may be a haptic display, where each of the actuators 618 (only one is numbered) may apply a pressure on the skin of the user's body part 604. The pressure pattern applied on the user's skin by the haptic display represents an output from the training environment, e.g. a present status of the game. Thus, the processing circuitry translates the intended motions into an input to the training environment where the input is transformed to an output to the haptic display, where the transformation depends on the task performed and the type of task. The pressure pattern applied on the user's skin by the haptic display comprising the actuators 618 may correspond to the pixel values of the display 614. Thus, locations of elements shown on the display may be represented by one or more pressure points in the haptic display.

The haptic display 616 is preferably firmly attached to the user's body part 604 to provide effective somatosensory stimulation.

In the above description of FIG. 6, the actuators 618 provides the somatosensory stimulation to a body part 604 adjacent to the affected body part 606. However, depending on the type of treatment, the somatosensory stimulation may be applied to the affected body part itself, and the sensors 618 may collect signals from the affected body part. This may for example be the case for treatment of stroke. Thus, the body part 604 may be an affected body part, whereby the electrodes 602 for collecting signals indicative of intended motions of the affected limb are placed on the affected body part. Further, the array 616 of actuators and/or electrodes are placeable on the user's affected body part for providing the somatosensory stimulation to the affected body part.

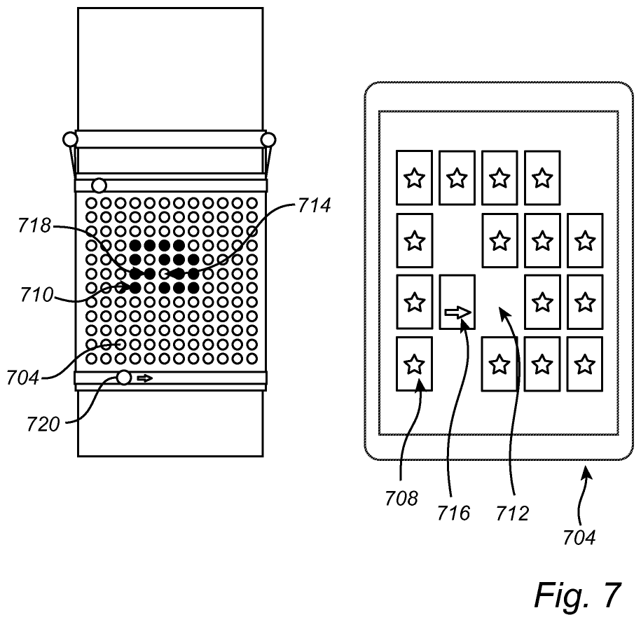

FIG. 7 is an example of a training environment 702 in the form of a game that could be part of a rehabilitation program for a user. The array 704 of actuators and/or electrodes that are placeable on the user's body part 706 for providing the somatosensory stimulation illustrates a translation of the training environment into haptic feedback stimuli. Here, the movable bricks 708 of the game is represented by a filled actuator 710 that is activated, for example the actuator 710 represents the brick 708. Empty positions, e.g. position 712 in the training environment 704 is represented by the empty actuator 714 being a not activated actuator. The user controls the motions of the bricks, e.g. here brick 716 is moved to the right, using an input system such as a system controlled by intended motions of an affected body part. Once the input provided by the user affects the brick 716 to move to the right into position 712, the haptic display 704 is caused to de-activate the actuator 718 and active the actuator 714 representing the new position of the brick 716. A slider 720 provides haptic feedback that represent the motion of the brick 716.

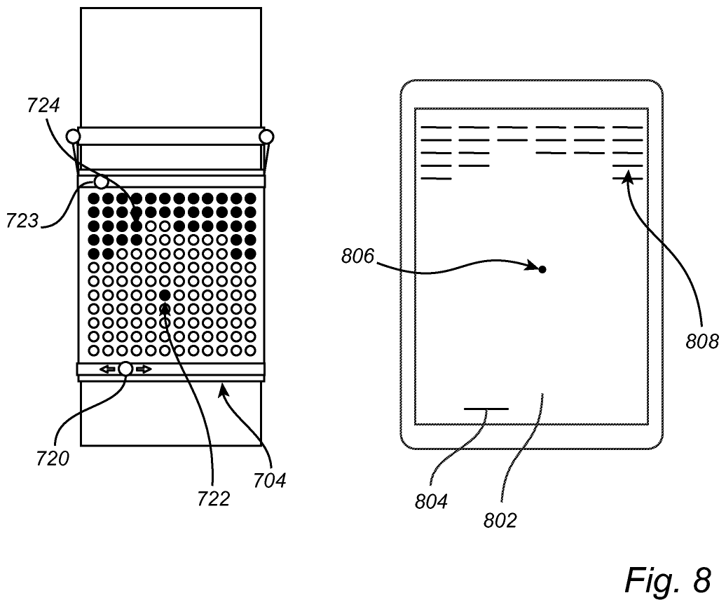

FIG. 8 is another example of a training environment 802 in the form of a game that could be part of a rehabilitation program for a user. The array 704 of actuators and/or electrodes that are placeable on the user's body part 706 for providing the somatosensory stimulation illustrates a translation of the training environment into haptic feedback stimuli.

The user controls the motion of the element 804 via an input system to move to the left and right to capture the “ball” 806. The motions and position of the element 804 is represented by the haptic feedback provided by the slider 720. The present position of the ball 806 in the training environment 802 is represented by activated actuator or electrode 722. The strips 808 that the user attempts to shoot down by deflecting or shooting the ball using the movable element 804 are represented by activated actuators or electrodes 724. Once the ball hits a strip 808 it disappears, and the corresponding actuators or electrodes are de-activated. The movement of the “ball” 806 can also be represented by another slider 723 at the top of the display so the bottom slider 720 represent the location of the movable element 804, and the top slider the location of the “ball” 806.

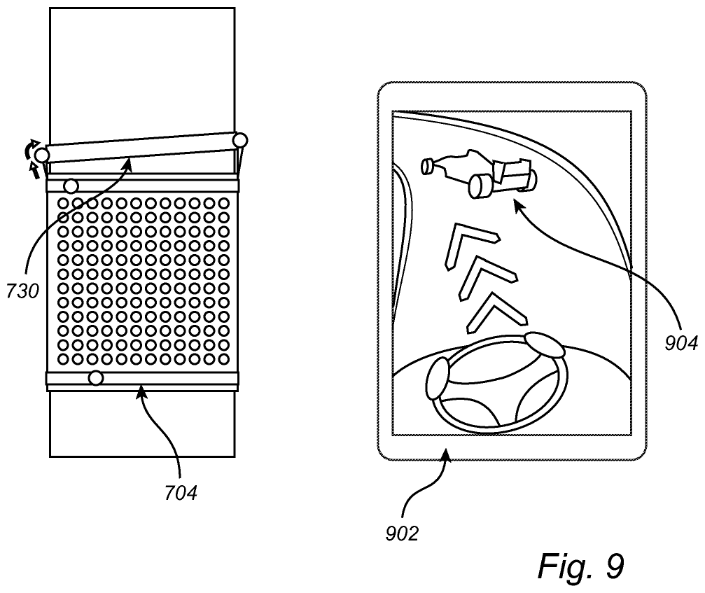

FIG. 9 is another example of a training environment 902 in the form of a game that could be part of a rehabilitation program for a user. In this example the user controls a racing car 904 along a track in the training environment 902 via an input system, e.g. controlled by muscle movements as discussed above. Haptic feedback that represents the position of the race car on the track and the steering motions is fed back to the user via the array 704 of actuators and/or electrodes as well as a stretch device 730 adapted to stretch or strain the skin to represent e.g. the degree that the steering wheel is turned or the curvature of the track.

In the examples illustrated in FIGS. 7-9 the training environments are shown on a visual and/or haptic displays.

Example variables of the training environments are e.g. locations, movements, sizes, and presence of the e.g. “balls”, bricks, and elements described in FIGS. 7-9. As a variable changes in response to user input, e.g. a brick 716 is moved, or a ball 806 moves or is deflected, or a strip 8 is removed, or a steering wheel is turned, an output is generated and provided to the user in the form of a sensory feedback including somatosensory feedback.

The somatosensory feedback provided to the user when e.g. moving an object in the training environment is one example of output being directly related to the input, this may be e.g. biofeedback.

Outputs related to consequences of the user input such as removal of an element or movement of the ball are example of output being indirectly related to the input.

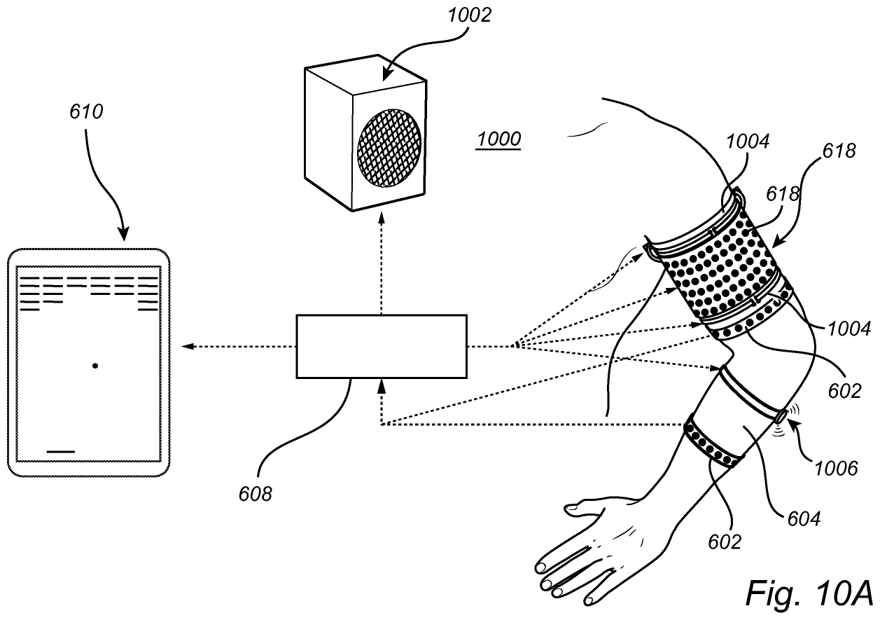

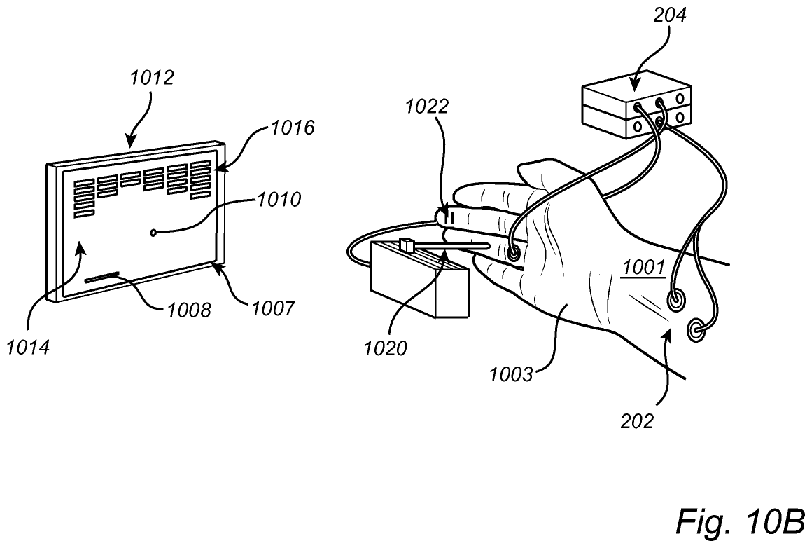

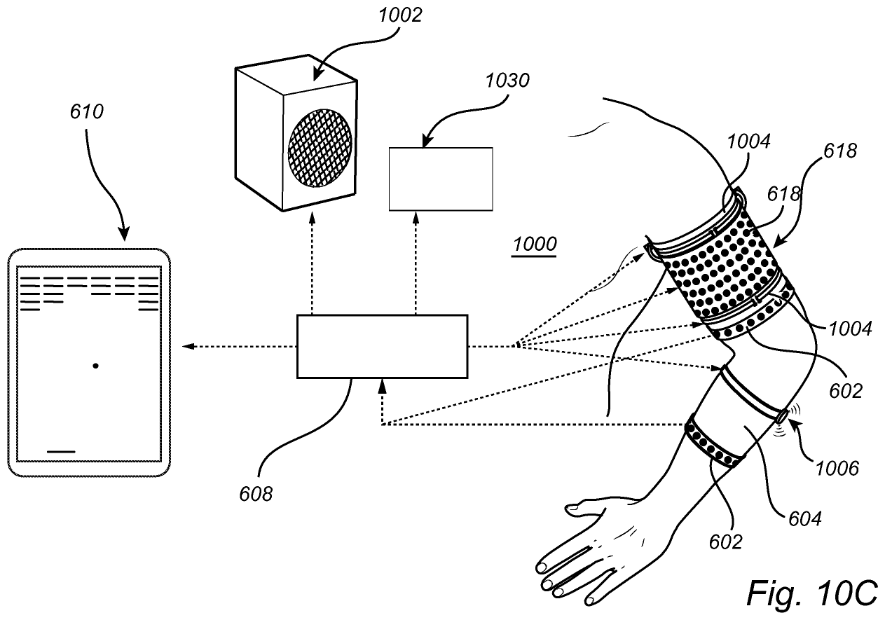

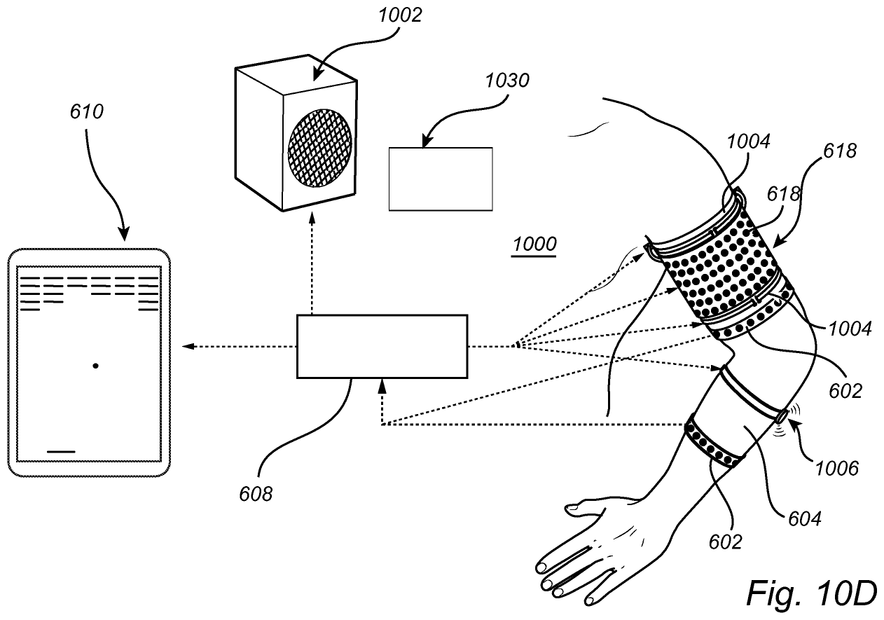

FIG. 10A illustrates a system 1000 according to embodiments of the present invention where a set of different output feedback systems are schematically illustrated. The system 1000 comprises the electrodes 602, the processing circuitry 608, and the array 616 of actuators 618 and/or electrodes discussed in conjunction with FIG. 6.

In addition, the system comprises an audio device 1002 for providing auditory feedback, haptic sliders 1004, and tendon vibrator 1006.

Variables from the training environment 610 of the task are translated into outputs that inform the user on their state. This translation can include but it is not limited to haptic, visual, auditory, or thermal representations such as: representing position and forces using tactile actuators 618, representing horizontal, vertical, or transversal movements of task objects via haptic sliders 1004 or tendon vibrator 1006, and/or vibration devices at contact and/or action with task objects.

The correct timing and consistency of the input/output translation is necessary to facilitate sensorimotor training, therefore any translation time is below the known human times of perceivable delays, i.e., around, or below 250 milliseconds.

Haptic feedback may be provided as tactile and electro-tactile stimuli combination as illustrated in FIG. 10B. In FIG. 10B, a set of electrodes 202 are used for collecting signals from the user's body part, here on the forearm 1001. The collected electromyography signals may be indicative of intended motions of the joints in the hand 1003 and provides for the user to control elements of the training environment 1007. For example, via the intended motions the user may control the horizontal location of the bar 1008 for capturing the ball 1010 when playing a game on the electronic device 1012, which also provides visual feedback via a display 1014. Here, the task in the training environment is to deflect the ball 1010 from the bar 1008 towards the bricks 1016 to have them removed as the ball hits the bricks.

Further, as output from the training environment, for example confirmation that a brick 1016 was successfully removed or that the ball 1010 deflected off the bar 1008, i.e. when the bar 1008 was successfully moved by the user so that the moving ball 1010 was captured, a mechanical actuator 1020 stimulates mechanoreceptors of the body part, here a finger, while an electrode 1022 stimulates the nerve producing distally referred sensations in the fingertip. Further, processing circuitry 204 is configured to operate a motor volition decoder for decoding the signals to infer the intended motions, and to control at least one element 1008 of the training environment 1014 based on the decoded signals. In other words, the processing circuitry is wirelessly, or via wires, able to receive the signals recorded by the sensors 202, decode the signals to infer the motions that the user intents to perform with an affected or missing body part.

In some possible implementations, the system 1000 comprises a brain modulation and/or brain stimulation device 1030 as conceptually illustrated in FIG. 10C. The brain modulation and/or brain stimulation device 1030 is configured to excite, inhibit, and/or stimulate the user's brain, in response to events in the training environment occurring while the user is performing a task, and/or prior to the task and/or after the task is completed, The brain modulation and/or brain stimulation device 1030 is here shown connected to the processing circuitry 608 to receive control signals therefrom that controls the provision of brain stimulation or modulation from the device 1030 to the user's brain. In this way, the brain modulation and/or brain stimulation device 1030 may receive triggering signals form the processing circuitry 608 indicative of for example triggering events in the training environment that activates the provision of the brain modulation and/or brain stimulation to the user. However, in some embodiments, the brain stimulation device 1030 provides the brain modulation and/or brain stimulation to the user independent of the training environment and in such case the brain stimulation device 1030 may operate in parallel with the processing circuitry, as conceptually illustrated in FIG. 10D. In such case, the brain modulation or brain stimulation may be provided to the user independent of the task performed by the user in the training environment.

Example devices 1030 that may provide brain modulation and/or stimulation, include tDCS, tACS, tRNS, tPCS, TMS, TES, or similar. Such devices are often worn on the user's or placed near the user's skull for providing the stimulation or modulation.

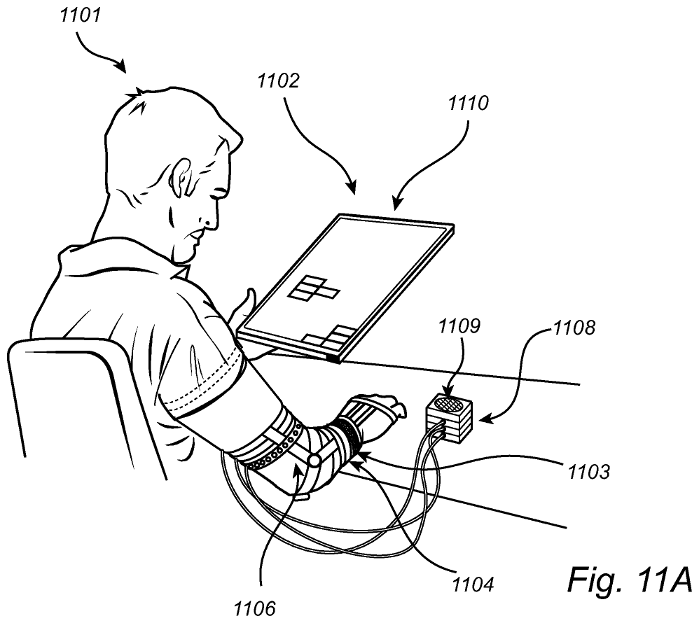

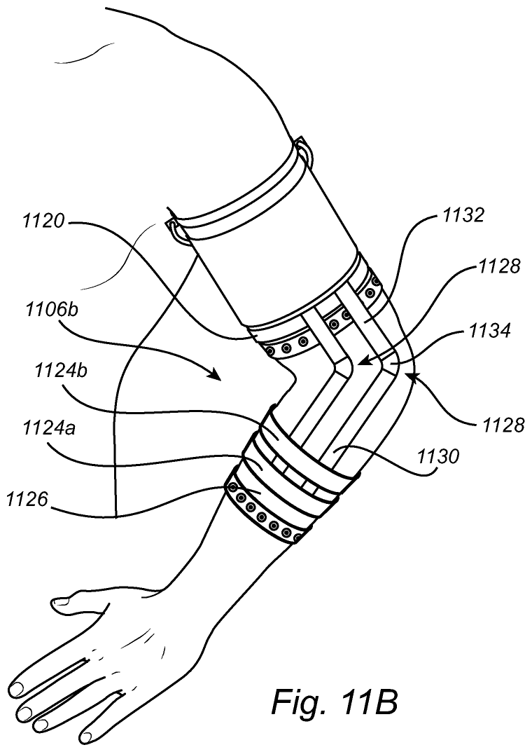

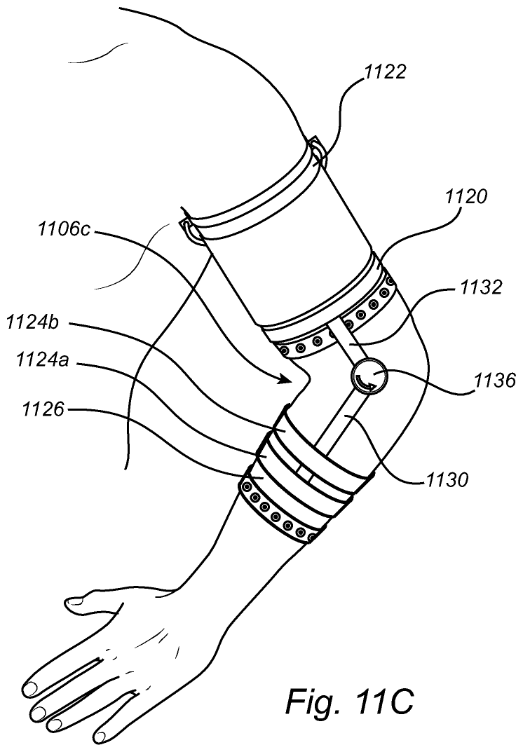

FIG. 11A conceptually illustrates a user 1101 using a system according to embodiments of the present invention. The system 1102 includes electrodes 1103 for collecting signals indicative of intended motions of an affected limb 1104. In this embodiment, the system comprises an exoskeletal stabilization unit 1106 arrangeable to stabilize the motion of the user's body part 1104. Processing circuitry 1108 receives and decodes signals from the electrodes 1102. The system may further comprise an audio device 1109 that provides auditory feedback to the user 1101 when performing the task in the training environment shown on a display 1110. Here in the form of playing Tetris®. The auditory feedback may enhance actions occurring on the display, e.g. successfully moving or placing a brick of the Tetris game.

The exoskeletal stabilization unit 1106 may be powered to assist the user in moving the body part when performing the task. In such case, the processing circuit may be configured to control an electric motor arranged to power the exoskeletal stabilization unit 1106, according to the decoded signals.

FIG. 11B and FIG. 11C conceptually illustrates two types of exoskeletal stabilization units 1106a and 1106b. The exoskeletal stabilization units comprise a first attachment member 1120 shaped as a collar adapted to be attached around the user's body part, here the upper arm 1122. The first attachment member 1120 may for example be strapped around the arm of the user. Further, the exoskeletal stabilization units comprise two lower attachment members 1124a-b attached to or around the user's lower arm 1126.

Turning now to FIG. 11B, conceptually illustrating a passive exoskeletal stabilization unit 1106b arrangeable to stabilize the motion of the user's part. In this embodiment, the exoskeletal stabilization unit 1106b comprises linkage arms 1128 for coupling the first attachment member 1120 to the lower attachment members 1124a-b. Thus, the linkage arms 1128 are connected to the first attachment member 1120 and to the lower attachment members 1124a-b. Further, each of the linkage arms comprises a first link 1130 and a second link 1132 joined at a joint 1134 that provides for a rotatable connection between the first link and the second link such that the user may stretch his/her elbow joint.

FIG. 11C conceptually illustrates an active exoskeletal stabilization unit 1106c that is powered to assist the user in moving the body part when performing the task. Here, the joint 1136 between the first link 1130 and a second link 1132 is provided with an electric motor that may assist the user in using his/her elbow joint. Thus, the joint 1136 may be electrically actuated such that the angle between first link 1130 and a second link 1132 at the joint 1136 is varied.

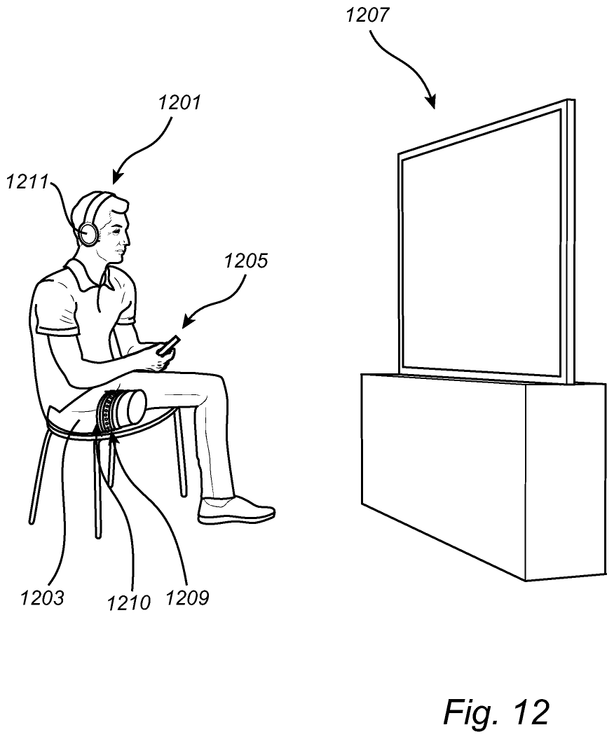

FIG. 12 conceptually illustrates a user 1201 using a system according to embodiments of the present invention. The user is here an amputee, thus missing a limb adjacent to the body part 1203 being the thigh. Thus, the user is using an input system in the form of a game controller 1205 for playing a game or performing a task in a training environment shown on the desktop display 1207. Motor volition of the missing limb can also be decoded using signals from electrodes 1209 in the thigh 1203 to be used as input for playing the game. Somatosensory stimulation may be provided via electrodes and/or actuators 1210 placed in contact with the body part 1209. Auditory feedback from the training environment may be provided using the headphones 1211.

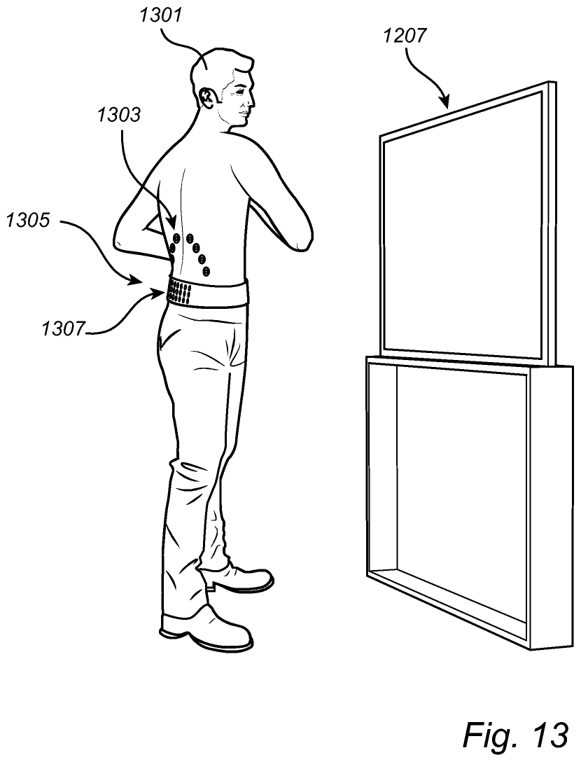

FIG. 13 conceptually illustrates a user 1301 using a system according to embodiments of the present invention. The user 1301 has sensing electrodes 1303 arranged on the users back 1305 for collecting signals indicative of intended motions, here in the form of turning the upper body. Visual feedback from the training environment is provided on the desktop display 1207, and somatosensory stimulation may be provided via electrodes and/or actuators 1307 placed in contact with the body part 1305 for treating e.g. back pain.

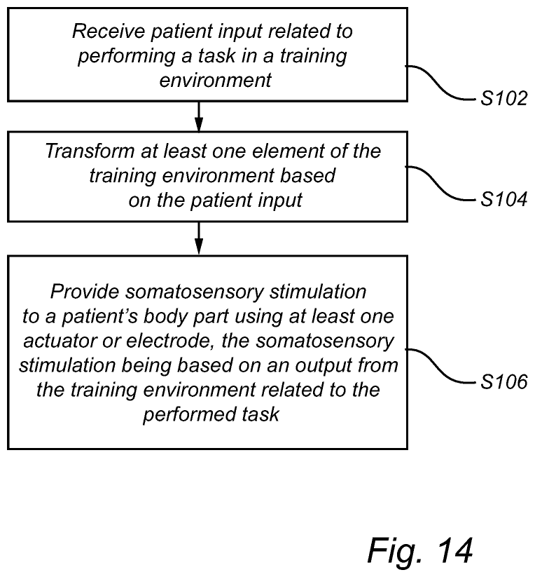

FIG. 14 is a flow-chart of method steps according to embodiments of the present invention. The method comprises step S102 of receiving user input related to performing a task in a training environment from an input system configured to provide control to a user for performing the task in the training environment. In step S104, transforming at least one element of the training environment based on the user input. In step S106, providing somatosensory stimulation to a user's body part using at least one actuator or electrode, the somatosensory stimulation being based on an output from the training environment related to the performed task.

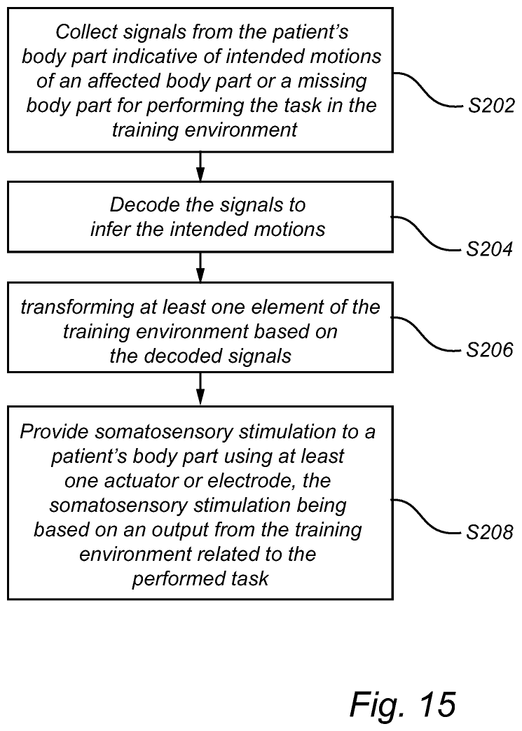

FIG. 15 is a flow-chart of method steps according to embodiments of the present invention. The method comprises step S202 of collecting signals from the user's body part indicative of intended motions of an affected body part or a missing body part for performing the task in the training environment. In step S204, decoding the signals to infer the intended motions. In step S206, transforming at least one element of the training environment based on the decoded signals. In step S208, providing somatosensory stimulation to the user's body part using at least one actuator or electrode, the somatosensory stimulation being based on an output from the training environment related to the performed task.

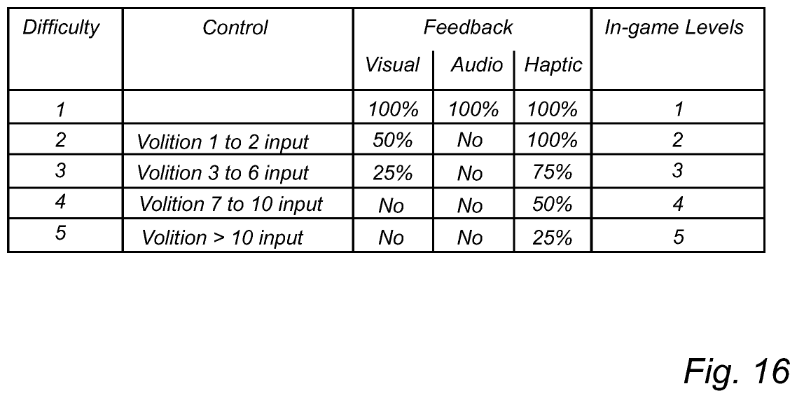

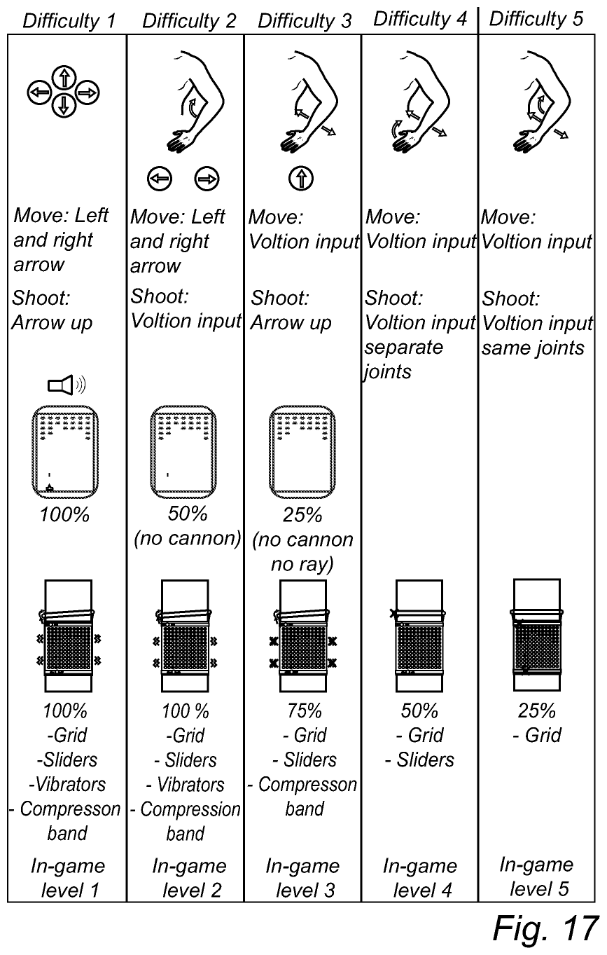

The method may comprise providing progressively more difficult tasks to the user depending on the outcome of the prior performed task. This is schematically illustrated in FIGS. 16 and 17.

The rehabilitation aspect herein relies on neuroplasticity to alleviate pain and restore function. Therefore, use of the invention ensures the continues engagement of the user's attention to the rehabilitation task at hand (e.g. mindful practice) by appropriately adjusting the difficulty level. Mindful practice is known to be a requisite to enable neuroplasticity.

Increasing difficult rehabilitation tasks are made available to the user through the progression in the therapy. Initial exercises are purely motor or sensory, and then simple close-loop control tasks in which only one movement and several types of sensory feedback are involved. The following rules are an example followed as the level of difficulty increases:
1. The types of sensory feedback, other than somatosensory, are reduced. This forces the users to relay more on somatosensory feedback.
2. The resolution of sensory stimulation increases. This requires higher skills of sensory discrimination to accomplish the task.
3. The number of movements to be executed for motor control increases. This requires the user to improve motor skills.
4. Simultaneous rather than only individual movements are required for motor control. This requires higher motor control skills.
5. Allowable margin of errors is reduced. This requires the user to provide more precise control and finer sensory discrimination.

The combination of the factors 1-5 above is adaptively changed on the user's performance. Table 1 in FIG. 16 shows an example in which the types of feedback are reduced as the level of difficulty increases from level 1 to level 5, while the number of control inputs is increased. This table exemplifies the reduction of feedback types, however, in parallel the resolution of haptic feedback can be increased to demand the user of higher discrimination skills (not illustrated).

For example, in difficulty level 1, input systems such as keyboard and mouse, and touchscreen is/are used, whereas full visual, auditory, and haptic feedback is provided as an output from the training environment.

In difficulty level 2, input systems relying on muscle movements, e.g. decoded intended motions where only 1 or 2 inputs are required is used, and only partial visual and full haptic feedback is used.

In difficulty level 3, input systems are used that rely on muscle movements, e.g. decoded intended motions where 3 to 6 different motions are used as inputs, and only partial visual and full haptic feedback is provided as output to the user.

In difficulty level 4, input systems are used that rely on muscle movements, e.g. decoded intended motions where 7 to 10 different motions are used as inputs, and partial haptic feedback is provided as output to the user.

In difficulty level 5, input systems are used that rely on muscle movements, e.g. decoded intended motions where more than 10 different motions are used as inputs, and even further reduced partial haptic feedback is provided as output to the user.

Accordingly, providing progressively more difficult tasks may correspond to starting at the difficulty level 1 and moving to higher levels depending on the performance of the user in performing the tasks.

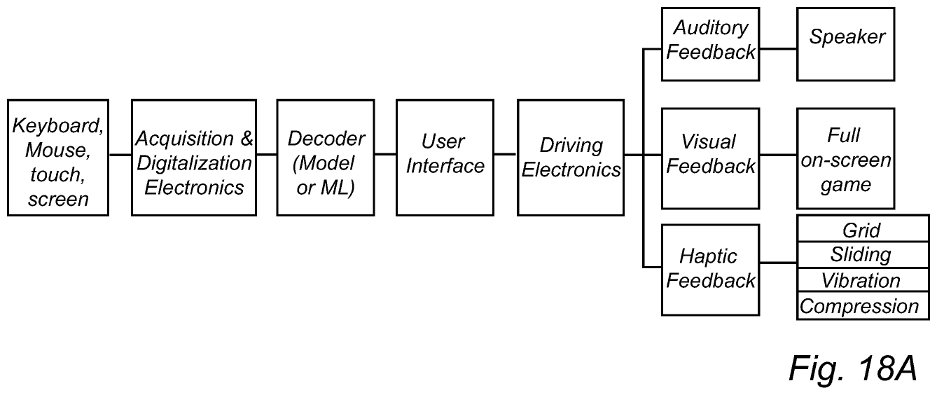

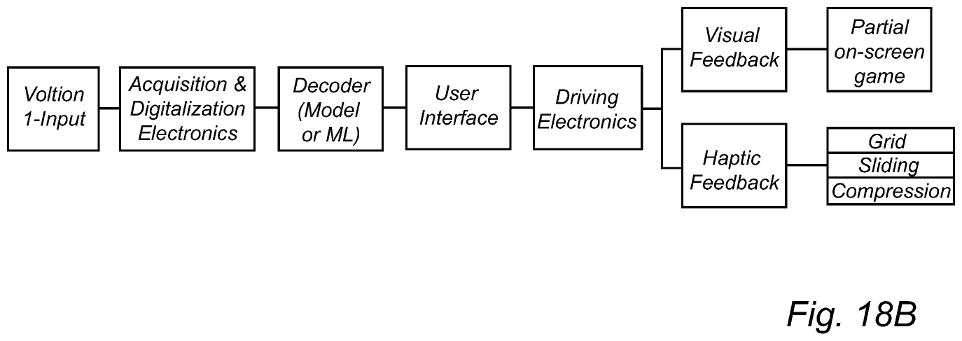

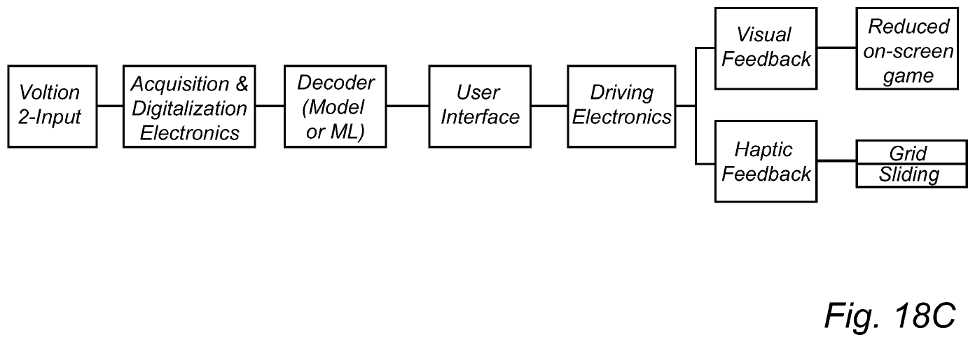

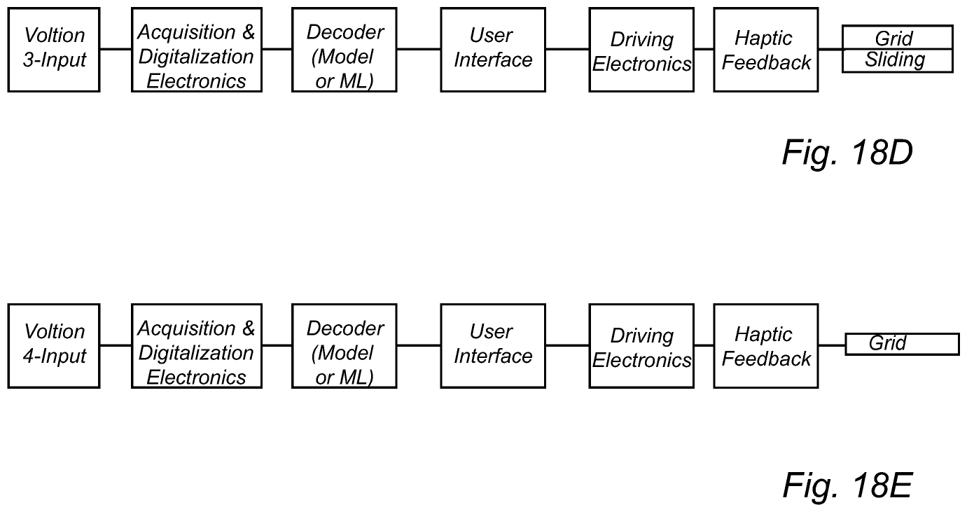

FIG. 17 schematically illustrates an example of a game in which all sensory types are provided in the lowest level of difficulty (level 1), and how this can be reduced to increase difficulty, e.g. levels 2-5. FIG. 18A-E schematically illustrates how the difficulty level is related to the various types of outputs provided as feedback to the user. FIG. 17 and FIG. 18A-E are discussed in conjunction.

For example, in difficulty level 1, FIG. 17 and FIG. 18A, arrow keys on a keyboard are used as inputs, and the output is auditory, full visual feedback, and haptic feedback is provided by an array of actuators, sliders, vibrators, and a compression band.

As the difficulty progresses to level 2, FIG. 17 and FIG. 18B, the input is provided by a combination of arrow keys, e.g. right and left key to move the cannon (see visual feedback in level 1), and motion volition input to shoot the cannon. The visual feedback is reduced by removing the cannon which position is now provided only by the haptic feedback by a slider and an actuator in the array. Further, haptic feedback is provided by the array of actuators, sliders, vibrators, and a compression band.

As the difficulty progresses to level 3, FIG. 17 and FIG. 18C, the input is provided by a combination of arrow keys, e.g. motion volition to move the cannon, and arrow up key to shoot the cannon. The visual feedback is reduced by removing the cannon and the ray produced by the cannon which positions are now provided only by the haptic feedback by a slider and actuators in the array. Further, haptic feedback is provided by the array of actuators, sliders, and a compression band.

As the difficulty progresses to level 4 FIG. 17 and FIG. 18D, the input is provided only by motion volition where motion volition in different joints may be used for moving the cannon and shooting the cannon. The visual feedback is removed, and only haptic feedback is provided as output from the training environment. The position of the canon and the ray is represented by actuators in the array and by sliders.

As the difficulty progresses to level 5 FIG. 17 and FIG. 18E, the input is provided only by motion volition but now using motion volition using the same joint both for moving the cannon and shooting the cannon. As output, only haptic feedback is provided from the training environment. The position of the canon and the ray is represented by actuators in the array.

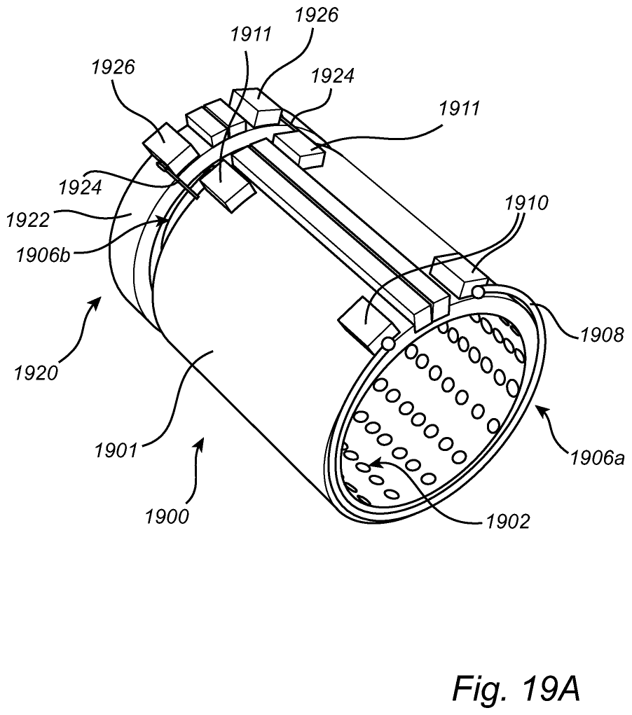

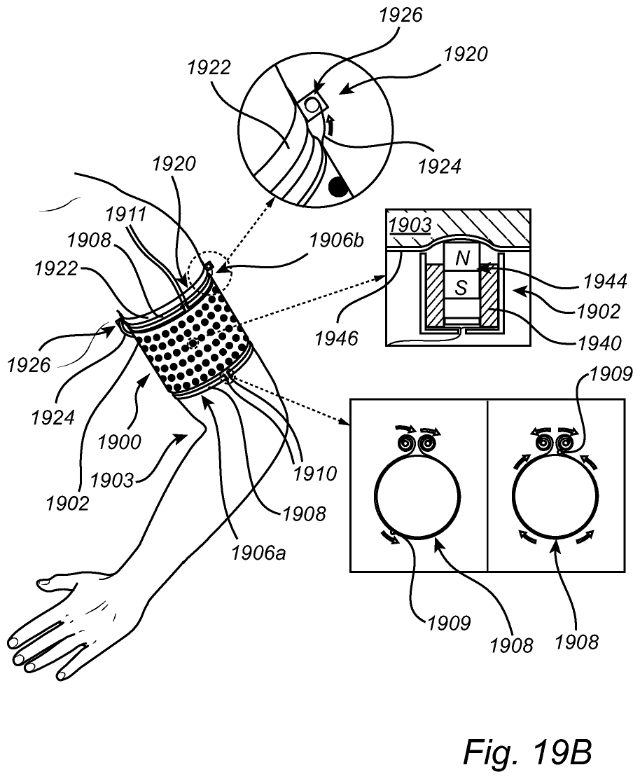

Embodiments of the present invention are adapted to active sensory receptors in various ways, and preferably with as few components as possible. FIG. 19A-B will be described in conjunction.

FIG. 19A conceptually illustrates an example haptic feedback arrangement 1900 adapted to be arranged around a body part such as a limb of a user, and FIG. 19B illustrates the haptic feedback arrangement 1900 attached to a user's upper arm 1903. The haptic feedback arrangement 1900 is shaped as a cuff 1901 or collar that is intended to wrap around the user's limb.

An array of actuators 1902 (only one is numbered) are located on the inner side of the haptic feedback device 1900, i.e. on the inner side of the cuff 1901, such that tactile stimulation can be provided to the skin of the user's body part.

Further, on distal ends of the cuff 1901 is a respective slider device 1906a,b arranged. The slider device 1906a will now be described, the slider 1906b comprises the same components as the slider device 1906b. The sliders 1906a comprises a rope or string 1908 that can be wrapped around the limb. The ends of the string 1908 are attached to a respective continuous servomotor 1910. By actuating both servo motors 1910 in the same direction, a sliding sensation (hair follicle plexus) on the skin of the user's body part is achieved. Adding e.g. a knot 1909, better seen in FIG. 19B, on the string 1908, contributes to the possibility of identifying a single position on the axis of the string. The servo motors of the slider device 1906b are denoted 1911.

Further, the haptic feedback arrangement 1900 comprises another sensory feedback device on the distal end of the cuff 1901 where the slider device 1906b is located. The sensory feedback device is here in the form of a stretch device 1920 adapted to stretch or strain the skin. The stretch device 1920 comprises a collar 1922 or band adapted to be attached and tightened around the limb of the user. Further, strings 1924 are connected to a servo motor 1926 on one side, here on the side of the collar 1922, and to a fixed point on the other side, here the side of the cuff 1901.

By actuating one of the servo motors 1926, the corresponding string 1924 is tightened whereby the corresponding side of the collar is moved towards the cuff 1901 thereby creating a sense of skin stretch (Ruffini's endings), creating the illusion of proprioception.

Further, a possible implementation for providing a tactile point is conceptually illustrated in one of the close-up views in FIG. 19B. An actuator 1902 comprises a coil 1940 with a magnetic core 1944. By actuating the coil, the magnetic core moves in and out of the coil 1940, creating a tactile point when it touches the skin 1946 of the user's body part 1903.

A control unit 608, see FIG. 6, may be configured to control a training environment 610 for functional rehabilitation and/or pain rehabilitation due to sensorimotor impairment, the control unit being configured to: receive user input related to the user performing a task in a training environment, the user input being receivable from an input system configured to provide control to a user for performing the task; control at least one element for affecting the task in the training environment based on the user input; process variables of the training environment effected by the performed task into an output to at least one actuator 618 or electrode placeable on/in the user's body for providing somatosensory stimulation.

The control unit may be configured to receive signals collected from a user's body part by a set of sensors, the signals being indicative of intended motions of an affected body part or a missing body part for performing the task in the training environment; decode the signals to thereby infer the intended motions, control the at least one element for affecting the task in the training environment based on the decoded signals.

There is further provided a computer program product comprising a computer readable medium having stored thereon computer program means for controlling a training environment for functional and pain rehabilitation due to sensorimotor impairment, wherein the computer program product comprises: code for processing user input related to performing a task in a training environment, the user input being receivable from an input system configured to provide control to a user for performing the task; code for controlling at least one element for affecting the task in of the training environment based on the user input; code for processing variables of the training environment effected by the performed task into an output to at least one actuator or electrode placeable on/in the user's body for providing somatosensory stimulation.

The computer program product may be operative on the control unit 608.

Processing circuitry or a control unit may include a microprocessor, microcontroller, programmable digital signal processor or another programmable device. The control unit may also, or instead, include an application specific integrated circuit, a programmable gate array or programmable array logic, a programmable logic device, or a digital signal processor. Where the control unit includes a programmable device such as the microprocessor, microcontroller or programmable digital signal processor mentioned above, the processor may further include computer executable code that controls operation of the programmable device.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which correspond to tangible media such as data storage media, or communication media including any media that facilitate the transfer of a computer program from one place to another, e.g., according to a communication protocol. In this manner, computer-readable media generally may correspond to (1) tangible computer-readable storage media which are non-transitory or (2) a communication media such as signal or carrier waves. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer.

Even though the invention has been described with reference to specific exemplifying embodiments thereof, many different alterations, modifications and the like will become apparent for those skilled in the art.

Additionally, variations to the disclosed embodiments can be understood and effected by the skilled person in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage.

1. A system for functional rehabilitation and/or pain rehabilitation due to sensorimotor impairment, comprising:
an input system for providing control to a user to perform a task in a training environment, the input system comprises a set of sensors placed on a user's body part, other than the head, adjacent to an affected body part or being the affected body part itself or adjacent a missing body part, for collecting signals indicative of intended motions of the affected body part or the missing body part to perform the task in the training environment, and a processing circuitry configured to operate a motor volition decoder for decoding the signals to infer the intended motions,
the system further comprises an array of actuators placed in contact with the user's body part for providing tactile stimulation to the user based on an output from the training environment related to the performed task,
wherein the processing circuitry is configured to control at least one element of the training environment based on the decoded signals.

2. The system according to claim 1, wherein the motor volition decoder is configured to decode the signals and infer the intended motions by the user according to a model.

3. The system according to claim 1, wherein the sensors are electrodes arrangeable on a user's skin for recording electric signals.

4. The system according to claim 1, wherein the sensors are electrodes that are implantable into the user's body for recording electric signals.

5. The system according to claim 1, further comprising an array of electrodes placeable on the user's body part for providing somatosensory stimulation.

6. The system according to claim 1, wherein the array of actuators provides a tactile representation of the training environment to the user.

7. The system according to claim 1, wherein the array of actuators is a haptic display.

8. The system according to claim 1, wherein the electrodes for providing the somatosensory stimulation comprises electrodes implantable into the user's body part.

9. The system according to claim 1, comprising kinesthetic feedback device configured to provide kinesthetic feedback to the user's body based on the outcome of the decoded signals.

10. The system according to claim 1, wherein the output from the training environment is directly or indirectly related to input provided by the user to the training environment for performing the task.

11. The system according to claim 1, comprising a display for providing visual feedback from the training environment.

12. The system according to claim 1, comprising an exoskeletal stabilization unit arrangeable to stabilize the motion of the user's body part.

13. The system according to claim 12, wherein the exoskeletal stabilization unit is powered to assist the user in moving the body part when performing the task.

14. The system according to claim 1, comprising an audio device for providing auditory feedback to the user based on a further output from the training environment.

15. The system according to claim 1, comprising a thermal device for providing thermal feedback to the user based on a further output from the training environment.

16. The system according to claim 1, wherein the training environment is a virtual training environment.

17. The system according to claim 1, comprising a brain modulation and/or brain stimulation device configured to excite, inhibit, and/or stimulate the user's brain.

18. The system according to claim 1, wherein the tasks is/are at least one of motor, sensory, and/or sensorimotor training tasks.

19. A control unit configured to control a training environment for functional rehabilitation and/or pain rehabilitation due to sensorimotor impairment, the control unit being configured to:
receive signals collected by a set of sensors placed on a user's body part, other than the head, adjacent to an affected body part or being the affected body part itself or adjacent a missing body part, the signals being indicative of intended motions of the affected body part or the missing body part for performing a task in a training environment, the user input being receivable from an input system comprises the set of sensors configured to provide control to a user for performing the task,
decode the signals to thereby infer the intended motions,
control at least one element for affecting the task in the training environment based on the decoded signals;
process variables of the training environment effected by the performed task into an output to an array of actuators placed in contact with the user's body part to provide tactile stimulation.

20. A computer program product comprising a non-transitory computer readable medium having stored thereon computer program means for controlling a training environment for functional and pain rehabilitation due to sensorimotor impairment, wherein the computer program product comprises:
code for processing user input related to performing a task in a training environment, the user input being receivable from signals collected by a set of sensors placed on a user's body part, other than the head, of an input system configured to provide control to a user for performing the task, the body part being adjacent to an affected body part or being the affected body part itself or adjacent a missing body part, the signals being indicative of intended motions of the affected body part or the missing body part for performing the task in the training environment;
code for decoding signals to thereby infer the intended motions;
code for controlling at least one element for affecting the task in of the training environment based on the decoded signals;
code for processing variables of the training environment effected by the performed task into an output to an array of actuators placed in contact with the user's body part to provide tactile stimulation.

21. A method for sensorimotor impairment rehabilitation comprising:
receiving user input related to performing a task in a training environment from an input system configured to provide control to a user for performing the task in the training environment, wherein receiving the user input comprises collecting signals by a set of sensor's placed on a user's body part, other than the head, the user's body part being adjacent to an affected body part or being the affected body part itself or adjacent a missing body part, the signals are indicative of intended motions of the affected body part or the missing body part for performing the task in the training environment
decoding the signals to infer the intended motions;
transforming at least one element of the training environment based on the decoded signals;
providing tactile stimulation to a user's body part using an array of actuators, the tactile stimulation being based on an output from the training environment related to the performed task.

22. The method according to claim 21, comprising providing progressively more difficult task to the user depending on the outcome of the prior performed task.

23. The method according to claim 21, comprising:
providing brain modulation or brain stimulation to the user while the user is performing a task in the training environment.

24. The method according to claim 21, comprising:
providing brain modulation or brain stimulation prior to the user performing a task in the training environment, or once the user is completed in performing a task in the training environment.

25. The method according to claim 21, comprising providing brain modulation or brain stimulation to the user independent of the task performed by the user in the training environment.

26. The method according to claim 21, the performed tasks is/are at least one of motor, sensory, and/or sensorimotor training tasks.
